(19) United States
(12) Patent Application Publication
(10) Pub. No.: US 2026/0033509 A1
(43) Pub. Date: Feb. 5, 2026

(54) DEVELOPMENT OF FOOD-GRADE ANTIMICROBIAL FUNGAL FERMENTATES USING YEAST EXTRACT, PROTEINS, OR AMINO ACIDS AS CULTURE SUBSTRATES

(71) Applicant: Wisconsin Alumni Research Foundation, Madison, WI (US)

(72) Inventors: Jaehyuk Yu, Madison, WI (US); Dasol Choi, Westwood, CA (US); Xingrui Fan, Madison, WI (US)

(21) Appl. No.: 19/286,032

(22) Filed: Jul. 30, 2025

Related U.S. Application Data
(60) Provisional application No. 63/677,053, filed on Jul. 30, 2024.

(51) Int. Cl. A23B 11/18; A23B 11/65; A23B 11/67; A61K 36/062; C12N 1/14

(52) U.S. Cl. CPC A23B 11/18; A23B 11/65; A23B 11/67; A61K 36/062; C12N 1/14

(57) ABSTRACT

The present invention provides antimicrobial compositions comprising an Aspergillus oryzae fermentate and methods of making said antimicrobial compositions. Also provided are products comprising the antimicrobial compositions as well as methods of using the compositions to prevent the growth of a microbe in or on a food product, surface, or subject or for use in skin care or hair growth.

### CROSS-REFERENCE TO RELATED APPLICATIONS

This patent application claims priority to U.S. Provisional Application No. 63/677,053, filed on Jul. 30, 2024, the contents of which are incorporated by reference in their entireties.

### BACKGROUND

Foodborne illness and the spoilage of food products continue to have a profoundly negative impact on public health and global food supplies. The Centers for Disease Control and Prevention (CDC) estimates that each year, 1 in 6 Americans (about 48 million people) get sick, 128,000 are hospitalized, and 3,000 die from eating contaminated food. The annual cost of foodborne illnesses in the United States is estimated at $15.6 billion. Listeria monocytogenes infections contribute to nearly 20% of these costs.

L. monocytogenes is a foodborne pathogen that is of concern to the dairy and meat industries due to its ubiquitous presence and high adaptability. L. monocytogenes can grow under refrigerated and acidic conditions and is resistant to osmotic shock. L. monocytogenes is also highly tolerant to desiccation and can quickly form sanitation-resistant biofilms on equipment surfaces.

Staphylococcus aureus is another significant cause of foodborne disease and is responsible for an estimated 241,000 illnesses per year in the United States. S. aureus is notorious for its ability to become resistant to antibiotics. Infections caused by antibiotic-resistant strains often occur in epidemic waves initiated by one or a few successful clones. S. aureus releases enterotoxins that cause toxic shock-like syndromes and are implicated in food poisoning, allergic diseases, and autoimmune diseases. Certain Salmonella strains and coliforms such as Escherichia coli O157:H7 can also lead to severe diseases and even fatalities when contaminated food is ingested by humans.

Unfortunately, the misuse of antibiotics has resulted in a dramatic rise of microbes that are resistant to both antibiotics and food processing/preservation methods. Thus, in the absence of the discovery of new antibiotics, human mortality rates from infectious diseases are predicted to rise more than tenfold by 2050. Methicillin-resistant Staphylococcus aureus (MRSA) and Candida albicans pose a particular threat, as there are an estimated 80,000 MRSA infections and 46,000 Candida infections annually in the United States. Pseudomonas aeruginosa, Klebsiella pneumoniae, Acinetobacter baumannii, and Enterococcus faecium strains are also commonly found to carry antibiotic-resistant genes that may nullify the available medications. Combined with E. coli and S. aureus, they are known as the ESKAPE pathogens.

Thus, there is a need in the art for antimicrobial compositions that can inhibit or prevent the growth of microbes, including those that cause foodborne illnesses and those that are antibiotic-resistant.

### SUMMARY

In a first aspect, the present invention provides methods of producing an antimicrobial composition. The methods comprise: (a) inoculating a medium with an Aspergillus oryzae strain to produce an inoculated medium; and (b) culturing the inoculated medium to produce a fermentate. In these methods, the medium comprises a source of amino acids at a concentration of 3-40 g/L and does not comprise casein. In some embodiments, the methods further comprise: (c) filtering the fermentate to obtain a cell-free fermentate filtrate.

In a second aspect, the present invention provides antimicrobial compositions produced by the methods described herein. In some embodiments, the antimicrobial composition is food grade.

In a third aspect, the present invention provides products comprising the antimicrobial compositions described herein. In some embodiments, the product is a food product. In other embodiments, the product is a medical device or composition.

In a fourth aspect, the present invention provides methods of killing or inhibiting the growth or proliferation of a microbe. In some embodiments, the methods are used to inhibit the growth of a microbe in or on a food product. In other embodiments, the methods are used to inhibit the growth of a microbe on a surface. In still other embodiments, the methods are used to inhibit the growth of a microbe in or on a subject.

In further aspects, the antimicrobial compositions provided herein may be used in soap, shampoo, lotions, or another personal consumer product. These products may be used to improve hair or skin via a mechanism other than antimicrobial activity.

### BRIEF DESCRIPTION OF THE DRAWINGS

FIG. 1 shows identification of yeast extract as the essential and sole element for NP1 antibacterial activity. Bacterial growth curves of (a) Staphylococcus aureus S-6, (b) Listeria monocytogenes 10403S, (c) Methicillin-resistant S. aureus (MRSA) OC 11521, and (d) Escherichia coli 1-894-1 in tryptic soy broth (TSB) medium mixed with different heat-treated NP1 fungal fermentates obtained from growing Aspergillus oryzae NRRL 3483 in culture broths lacking one component of the NP1 medium (i.e., NP1 without maltose or NP1 without yeast extract) or comprising only a single ingredient of the NP1 medium (i.e., malt extract only, maltose only, glucose only, yeast extract only). Bacterial proliferation was measured at 37° C. for 24 hours, and optical density at a wavelength of 600 nm (OD600) was checked every 30 minutes using a Bioscreen C.° system. Original TSB medium was used as negative control (Control). Note that yeast extract is necessary and sufficient for the antimicrobial activity of NP1 fungal fermentates.

FIG. 2 shows response surface methodology (RSM) models that were used to evaluate the effects of substrate concentration (in g/L), incubation time (in days), and inoculation level (in log conidia/mL) on the antimicrobial strength of NPy fermentates produced by culturing Aspergillus oryzae NRRL 3483 at 30° C. and 220 rpm. The zone of inhibition (ZOI) (diameter in mm) against Staphylococcus aureus S-6 was produced using 6-mm antimicrobial susceptibility test disks loaded with 100 μL of 20× extract of NP fermentate. (a) Response surface evaluating the combined effect of substrate concentration and incubation time on the antimicrobial strength of NPy fermentate. (b) Response surface evaluating the combined effect of inoculation level and incubation time on the antimicrobial strength of NPy fermentate. (c) Response surface evaluating the combined effect of substrate concentration and inoculation level on the antimicrobial strength of NPy fermentate. (d) The interaction plot evaluates the effects of inoculation level, substrate concentration, and inoculation level on the antimicrobial strength of NPy fermentate.

FIG. 3 shows growth curves of Gram-positive bacteria in TSB reconstituted with different concentrations of NPy solutions. OD600 was tracked every 30 minutes over a 24-hour incubation at 37° C. The lines represent the average optical density of a technical triplicate. (A) Growth curves of 11 different Staphylococcus aureus isolates. Isolate names are indicated at the top of the graphs. The isolates with their strain codes underlined are known to be methicillin-resistant. (B) Growth curve of Listeria monocytogenes 10403S in TSB media reconstituted with different percentages of NPy.

FIG. 4 shows growth curves of Gram-negative bacteria in TSB reconstituted with different concentrations of NPy solutions. OD600 was tracked every 30 minutes over a 24-hour incubation at 37° C. The lines represent the average optical density of a technical triplicate. (A) Growth curves of Pseudomonas aeruginosa strains 65, 2638, and 3060 (indicated on the top of the graphs). (B) Growth curves of Escherichia coli strains 1-894-1 and 2671. (C) Growth curves of Klebsiella pneumoniae strains 81-1269A and BAA-2146. (D) Growth curve of Salmonella enterica serovar Typhimurium strain S9.

FIG. 5 shows a growth curve of the opportunistic pathogenic yeast Candida albicans SC5314 in TSB reconstituted with different concentrations of NPy solutions. OD600 was tracked every 30 minutes over a 24-hour incubation at 37° C. The lines indicate the average optical density of a technical triplicate.

FIG. 6 shows the results of evaluating the bactericidal effect of NPy against enterohemorrhagic Escherichia coli O157:H7 using the plate-count method. Mueller Hinton broth reconstituted with water (MHB+Water, NPy level=0), NPy fermentate (MHB+100% NPy), and a 50% v/v NPy solution (MHB+50% NPy) were used as substrates for bacterial growth (n=1). E. coli strain FRIK 47 was inoculated into the substrates at timepoint 0, targeting a concentration of 5×10⁵ colony forming units (CFU) per milliliter of substrate, and incubated at 37° C. At sampling timepoints, 100 μL of the bacterial cultures were serially diluted and plated on tryptic soy agar (TSA) to enumerate the bacteria.

FIG. 7 shows an assessment of the antifungal properties of NPy against Penicillium roqueforti and Penicillium expansum PE-21. NPy fermentate (100%, without water) was diluted with reverse osmosis water (0%, without NPy) into 10, 25, 50, and 75% solutions, which were used to reconstitute 39 g/L of potato dextrose agar (PDA) powder to make the agar plates. The spore suspensions of both species were diluted, and 10 μL of the dilutions were spot-plated on the agar media to deliver inocula of 10, 10², 10³, and 10⁴ spores, along with phosphate saline buffer (inoculum of 0) as a negative control. The photos were taken after a 5-day incubation at 25° C.

FIG. 8 shows colony-forming units (CFU) of log-phase (actively growing) bacteria grown in TSB liquid medium generated using heat-treated NPc as a solvent. (a) Staphylococcus aureus, (b) Methicillin-resistant S. aureus, and (c) Listeria monocytogenes. The numbers of live bacterial cells were measured after 18 hours of incubation at 37° C. by counting log CFU/mL. The original TSB medium was used as a negative control. In all cases, 10⁵/ml of log-phase bacterial cells were initially used.

FIG. 9 shows the colony-forming unit (CFU) of log-phase bacteria grown in TSB liquid medium generated using heat-treated NPc as a solvent. (a) Escherichia coli, (b) Salmonella enterica serovar Typhimurium, (c) Klebsiella pneumoniae, and (d) Pseudonomas aeruginosa. The numbers of live bacterial cells were measured after 18 hours of incubation at 37° C. by counting log CFU/mL. The original TSB medium and PBS buffer were used as a negative control. In all cases, 10⁵/mL of log-phase bacterial cells were initially used. NPc+TSB: TSB liquid medium generated using heat-treated NPc as a solvent. NPc−TSB: heat-treated NPc without added nutrition.

FIG. 10 shows the results of evaluating the bactericidal effect of NPc using the plate-count method. Mueller Hinton broth reconstituted with water (NPc level=0) or NPc fermentate (NPc level=100) was used as the substrate for bacterial grow (n=3). Bacteria were inoculated into the substrates at timepoint 0, targeting a concentration of 5×10⁵ colony forming units (CFU) per milliliter of substrate, and incubated at 37° C. At sampling timepoints, 100 μL of the bacterial cultures were serially diluted and plated on TSA to enumerate the bacteria. The data points represent the average counts of biological triplicates, and the error bars represent the standard errors. (A) The change of methicillin-resistant Staphylococcus aureus OC 11521 density over time. (B) The change of Escherichia coli O157:H7 FRIK 47 density over time. (C) The change of Salmonella enterica serovar Typhimurium density over time.

FIG. 11 shows an assessment of the antifungal activities of NPc against mold species. (a) Illustration of the experiment layout on a 24-well plate. NPc solutions were produced by diluting NPc fermentate (100% NPc) with reverse osmosis water (0% NPc) to 20, 40, 60, and 80%. Potato dextrose broth powder was diluted in each concentration of NPc media at a concentration of 24 g/L. Each column of the 24-well plate was loaded with 1.5 mL of the same concentration of diluted NPc, and each row of the 24-well plate was inoculated with the same strain of fungus at a concentration of 10³ conidia/mL of media. (b) 24-well plate inoculated with Aspergillus fumigatus strains Af293 (row 1), CEA-10 (row 2), CEA-17 (row 3), and F16216 (row 4). Photo was taken after a 3-day incubation at 37° C. (c) 24-well plate inoculated with Penicillium species P. commune (row 1), P. chrysogenum (row 2), P. roqueforti (row 3), and P. expansum PE-21 (row 4). The photo was taken after a 5-day incubation at 25° C.

FIG. 12 shows the results of an antifungal disk diffusion assay against A. fumigatus strains. (a) The diameter of the clearance zone for eight A. fumigatus strains on Mueller Hinton agar (n=3). Amphotericin B (60 μg, AmB) and itraconazole (20 μg, ITAZ) were included as references, along with a solvent-only negative control (Neg Ctrl). (b) Representative images from the disk diffusion assay on potato dextrose agar plates showcase the vigorous antifungal activity of NPc and NPy ethyl acetate extracts compared to the reference drugs.

FIG. 13. shows the zone of inhibition diameters against Staphylococcus aureus S-6 produced by 6-mm antimicrobial susceptibility test disks loaded with 100 μL of 20× extract of NPc and NPy (n=16). Individual data points are shown in triangles and represent a biological replicate out of 16 batches. The bars extended away from the boxes represent the minimum and maximum data points. The top edge of the box represents the 75th percentile of the 16 data points, the bottom edge of the box represents the 25th percentile of the 16 data points, and the horizontal line in the middle of the box represents the median (50th percentile) of the 16 data points.

FIG. 14 shows the zone of inhibition diameters against Staphylococcus aureus S-6 produced by 6-mm antimicrobial susceptibility test disks loaded with 100 μL of 20× extract of NPew. Data for five biological replicates are shown.

FIG. 15 shows an evaluation of the effect of using different food and nutritional products as the substrate (at a concentration of 20 g/L) to produce antimicrobial NP fermentates using Aspergillus oryzae NRRL 3483. The zone of inhibition diameters against Staphylococcus aureus S-6 were produced by 6-mm antimicrobial susceptibility test disks loaded with 100 μL of 20× extract of the indicated NP fermentate.

FIG. 16 shows the antimicrobial potencies of NPc, NPew, and NPy fermentates produced by Aspergillus oryzae strains NRRL 3483, NRRL 3484, and NRRL 32657 (n=2). The zone of inhibition diameters against Staphylococcus aureus S-6 were produced by 6-mm antimicrobial susceptibility test disks loaded with 100 μL of 20× extract of corresponding NP fermentates.

FIG. 17 shows an assessment of the heat-stabilities of NPc, NPew, and NPy by disk diffusion assay (n=2). NPc, NPew, and NPy fermentates were sterilized by using 0.45-μm syringe filters (Filter-sterilized) or using a liquid autoclave cycle at 121° C. and 15 psig for 20 minutes (Autoclaved). The zone of clearance diameters against Staphylococcus aureus S-6 were produced by 6-mm antimicrobial susceptibility test disks loaded with 100 μL of 20× extract of the indicated NP fermentate. The error bars indicate the standard error of biological duplicates.

FIG. 18 shows that NPc cheese coating reduces Listeria monocytogenes on cheddar cheese surfaces. NPc percentage represents the concentration used to dissolve xanthan gum (5 g/L) relative to the original NPc fermentate, with 100% being only NPc, 50% being one part NPc diluted with one part DI water, and the negative control being only DI water.

FIG. 19 shows that NPc cheese coating inhibits Penicillium growth on cheddar cheese surfaces. NPc cheese coating solution contains the same amount of antimicrobial content as the original NPc fermentate. Xanthan gum-thickened DI water was used as a negative control. Pictures were taken every week for cheese samples stored at 12° C., inoculated with (A) P. roqueforti, (B) P. camemberti, (C) P. chrysogenum, and (D) P. commune. (E) Mold growth (colony diameter) on cheese samples stored at 4° C. for five weeks.

FIG. 20 shows that NPc addition inhibits mold growth on yogurt surface while having a minimal impact on lactic acid bacteria. (A) Mold plate count in yogurt with freeze-dried NPc powder at ˜1.1% w/w. (B) Streptococcus thermophilus plate count in untreated and NPc-treated yogurts. (C) Lactobacilli plate count in untreated and NPc-treated yogurts. (D) Mold growth photos in untreated and NPc-treated yogurts grown at 4° C. for five weeks. In each plate: top-left=Mucor circinelloides, top-right=Penicillium commune, middle-left=yogurt isolate Penicillium sp., middle-right=Penicillium roqueforti, bottom-left=uninoculated control, and bottom-right=Penicillium chrysogenum.

### DETAILED DESCRIPTION

The present invention provides antimicrobial compositions comprising an Aspergillus oryzae fermentate and methods of making said antimicrobial compositions. Also provided are products comprising the antimicrobial compositions as well as methods of using the antimicrobial compositions to prevent the growth of a microbe in or on a food product, surface, or subject.

The present inventors have created A. oryzae fermentates that exhibit strong antimicrobial activities against bacteria and fungi. Their fermentates are cell-free and are produced by culturing A. oryzae in a culture medium for several days and then filtering out all fungal cells.

In previous work, the inventors characterized the antimicrobial effects of two fermentates, referred to as natural preservative 1 (NP1) and natural preservative 2 (NP2). NP1 was produced by culturing A. oryzae in NP1 medium, which comprises malt extract, maltose, dextrose, and yeast extract, and NP2 was produced by culturing A. oryzae in NP2 medium, which comprises pancreatic digest of casein, papaic digest of soybean, dextrose, sodium chloride, and dipotassium phosphate. However, the inventors discovered that pancreatic digest of casein is the only ingredient of NP2 medium that is necessary to produce a fermentate with antimicrobial activity. They found that fermentate produced using a medium comprising only pancreatic digest of casein, which is referred NPc, has similar or even greater antimicrobial activity as compared to NP2. This previous work is described in International Patent Application Publication No. WO2023/102065, which is hereby incorporated by reference in its entirety.

In the work described in the present application, the inventors further discovered that yeast extract is the only ingredient in NP1 medium that is necessary to produce a fermentate with antimicrobial activity. The fermentate produced using a medium comprising only yeast extract is referred to as NPy. The inventors found that by using as little as 3.2 g/L of yeast extract in the medium, they can produce a fermentate with strong antimicrobial activity (see Example 1). Additionally, the inventors identified several strains of A. oryzae (see Example 3) and several additional sources of amino acids (see Example 4) that can be used to produce fermentates with antimicrobial activities.

The A. oryzae fermentates disclosed herein offer several advantages over other antimicrobial agents. First, the fermentates are produced by culturing a generally recognized as safe (GRAS) fungus (i.e., A. oryzae) in an edible culture medium, so they are expected to be safe for use in the food, medical, and pharmaceutical industries. Second, the inventors have demonstrated that the antimicrobial activities of their fermentates are heat stable up to at least 121° C. (see Example 6), which will allow them to be subjected to thermal processing. Third, because the antimicrobial activity of the fermentates is strong, they can be used effectively at low levels, which may help to prevent the development of microbial tolerance. Fourth, because filamentous fungi offer greater secretory capacity than bacteria and yeast, the fermentates are expected to be easily produced at commercial scales at low cost.

### Methods of Making Antimicrobial Compositions

In a first aspect, the present invention provides methods of producing an antimicrobial composition. The methods comprise: (a) inoculating a medium with an Aspergillus oryzae strain to produce an inoculated medium; and (b) culturing the inoculated medium to produce a fermentate. In these methods, the medium comprises a source of amino acids at a concentration of 3-40 g/L and does not comprise casein.

An “antimicrobial composition” is a composition that kills or inhibits the growth of one or more microbes. A “microbe” or “microorganism” is an organism of microscopic size. Microbes include bacteria, archaea, and certain types of eukaryotes, such as microscopic fungi, protists, rotifers, and unicellular plants (e.g., algae). A microbe may be unicellular or multicellular.

The term “inoculating” refers to the act of introducing a microbe (in this case, A. oryzae) into a composition. A microbe may be introduced into a composition by simply contacting the composition with some amount of the microbe. An “inoculated medium” is a medium into which at least microbe has been introduced. In some embodiments, the medium is inoculated with about 10³ to 10⁷ conidia/mL of A. oryzae, preferably at least 10⁴ conidia/mL. The number of A. oryzae conidia in a substance can be determined using standard methods known in the art, including methods that utilize a hemocytometer and/or a Neubauer chamber.

The term “Aspergillus oryzae strain” refers to any strain or isolate of the species A. oryzae. A. oryzae is a koji mold that is commonly used in the production of fermented foods and beverages, such as sake, shōchū, soy sauce, and miso. It is characterized as a generally recognized as safe (GRAS) substance by the United States Food and Drug Administration (U.S. FDA). In Example 3, the inventors identify three strains of A. oryzae (i.e., NRRL 3483, NRRL 3484, and NRRL 32657) that can be cultured in a medium comprising yeast extract (i.e., NPy medium) to produce an antimicrobial fermentate. In Example 5, they demonstrate that these strains can also be cultured in a medium comprising dried egg white protein (i.e., NPew medium) to produce an antimicrobial fermentate. Thus, in some embodiments, the A. oryzae strain used in the method is NRRL 3483, NRRL 3484, or NRRL 32657. Further, in Example 3, the inventors identify 27 additional A. oryzae strains (i.e., NRRL 506, NRRL 6270, KACC SD20, KACC 46922, KACC Aor44, KACC SD45, Han AO097, KACC 46923, KACC Meju 529, Han #6, KACC 46465, KACC 46456, KACC 46457, KACC 93120 (M385), KACC 46466, KACC 46920, KACC 46891, Han AO2014, Han #334, KACC 46810, KACC 46468, China 3.267, China 3.47, KACC M1017, Han #266, Han #17, or Han #13) that can be used to produce cell-free extracts with antimicrobial activity (see Table 11). Thus, in other embodiments, the A. oryzae strain used in the method is NRRL 506, NRRL 6270, KACC SD20, KACC 46922, KACC Aor44, KACC SD45, Han AO097, KACC 46923, KACC Meju 529, Han #6, KACC 46465, KACC 46456, KACC 46457, KACC 93120 (M385), KACC 46466, KACC 46920, KACC 46891, Han AO2014, Han #334, KACC 46810, KACC 46468, China 3.267, China 3.47, KACC M1017, Han #266, Han #17, or Han #13.

As used herein, the term “culture” refers to a composition comprising at least one microbe and factors needed for the growth, reproduction, and/or replication of that microbe. Typically, these factors are provided in the form of a culture medium. (See the section below titled “Culture medium” for a detailed description of this term.) The term “culture” encompasses compositions into which the microbe has just been added, as well as compositions into which the microbe has been allowed to grow for a period of time.

As used herein, the term “culturing” refers to a method in which a microbe is grown in a culture. Culturing involves providing essential nutrients to the microbe, maintaining conditions (e.g., temperature, humidity, airflow, barometric pressure, percent oxygen, percent carbon dioxide) that are suitable for growth and reproduction of the microbe, and waiting for a period of time to allow the microbe to grow and reproduce. For example, in some embodiments, culturing is performed at about 22° C. to about 30° C. In some embodiments, culturing involves agitating or shaking the culture at about 150 to 220 revolutions per minute (rpm). In some embodiments, the culturing is performed for about 1-11 days, 2-10 days, 4-9 days, or 6-8 days. In Example 1, the inventors estimated that the optimal incubation time for NPy is about 6.78 days based on a response surface methodology (RSM) experiment. Thus, in preferred embodiments, the inoculated medium is cultured for 6-7 days.

A “fermentate” is a product that has undergone fermentation. The fermentates of the present invention are produced by culturing an A. oryzae strain in a medium for a period of time. As the A. oryzae strain grows in culture, it breaks down substances in the culture medium (i.e., it ferments the medium). The A. oryzae strain may also secrete factors such as carbon dioxide, ethanol, amylase, fumarate, lactic acid, citric acid, itaconic, and malate into the culture medium. Thus, as used herein, the term “fermentate” refers to a medium that has been altered in these ways by the presence of A. oryzae.

In some embodiments, the methods further comprise: (c) filtering the fermentate to obtain a cell-free fermentate filtrate. As used herein, the term “filtering” means to pass a substance through a filter. A “filter” is a porous physical barrier that is permeable some substances but impermeable to others due to their physical size. The term “filtrate” refers to the portion of a substance that passes through the filter. Any filter with a pore size of 0.22 μm or smaller can be used to remove bacteria, yeast, and fungi from the fermentate. As described in Example 1, the inventors filtered their NPy fermentate through a Whatman Grade GF/A glass microfiber filter. Thus, in some embodiments, the filter is a glass fiber filter. In some embodiments, the filtering step comprises passing the fermentate through two or more filters with different pore sizes. Other suitable filters that can be used in the methods of the present invention include, but are not limited to, Miracloth, filter paper, and PES membrane filters (e.g., 0.22 or 0.45 μm). Filtering is not necessary to generate the antimicrobial activity of the fermentate.

In the methods of the present invention, the filtering step serves to remove living cells and other particles to enhance the stability and quality of the fermentate. Removing fungal cells (i.e., mycelia) from the fermentate produces a cell-free fermentate. As used herein, the term “cell-free” means lacking intact cells, i.e., cells in which the integrity of the plasma membrane is maintained. This is accomplished using a filter that is impermeable to cells (i.e., comprises pores smaller than cells). The removal of living cells creates a sterile composition. Thus, in some embodiments, the filtrate is a cell-free, sterile filtrate. Alternatively, the fermentate may be sterilized via extraction or using heat (e.g., via autoclaving, boiling, or subjecting the fermentate to a temperature greater than 100° C. for at least 5 minutes).

In some embodiments, the methods further comprise extracting the cell-free fermentate filtrate to obtain an extract of the cell-free fermentate filtrate. As used herein, the term “extracting” refers to a process of selectively removing a compound of interest from a mixture. An “extract” is the product of one or more extractions. Extraction methods are known in the art and include, for example, solvent extraction, filtration, membrane separation, chromatography, precipitation, distillation, and electrophoretic methods. Fermentate extracts were prepared using liquid biphasic extraction, but other methods may also be used.

In some embodiments, the extracting step comprises adding an extractant to the fermentate. As used herein, an “extractant” is a solvent that is used to physically separate a subset of solutes from a solution via a multi-phasic liquid separation technique. Examples of extractants include hexane, diethyl ether, ethyl acetate, ethanol, methanol, methyl acetate, acetone, toluene, dichloromethane, chloroform, tetrahydrofuran, “EMW” (i.e., a mixture of ethyl acetate, methanol, and water), and “CEF” (i.e., a mixture of chloroform, ethyl acetate, and formic acid). In some embodiments, the extractant is suitable for use in a biphasic liquid extraction. In some embodiments, the extractant is a polar solvent. In some embodiments, the extractant is an aprotic polar solvent.

In embodiments in which a biphasic liquid extraction is performed, the fermentate-extractant mixture is allowed to separate into immiscible layers based on the different densities of the fermentate and the extractant. Then, the extractant layer is separated from the rest of the mixture as a fermentate extract. In some embodiments, the extractant is added to the fermentate at an extractant:fermentate ratio of about 3:1, 2:1, 1:1, 1:2, or 1:3.

In some embodiments, the methods further comprise concentrating the extract to obtain an extract concentrate. As used herein, the term “concentrating” refers to a process of removing a solvent or another diluting agent from a solution. Concentration produces a “concentrate”, i.e., a solution comprising a higher concentration of at least one solute as compared to the original solution prior to the concentration step. In some embodiments, the extract is concentrated via drying. An extract may be dried via dehydration, lyophilization, convection air drying, or any other method available to those skilled in the art.

Methods in which the extract is concentrated may further comprise reconstituting the extract concentrate with a reconstitution solvent. The term “reconstituting” refers to a process in which a dried substance (e.g., a dried extract) is dissolved or diluted in a solvent. Accordingly, as used herein, the term “reconstitution solvent” refers to a solvent used to solubilize a dried extract. Any solvent in which the dried extract will dissolve may be utilized and should be selected based on the intended use for the antimicrobial composition. Examples of reconstitution solvents include, without limitation, methanol, ethanol, water, saline, drinkable beverages, ointments, and lotions.

In some embodiments, the steps of (1) extracting the cell-free fermentate filtrate to obtain an extract of the cell-free fermentate filtrate, (2) concentrating the extract to obtain an extract concentrate, and (3) reconstituting the extract concentrate with a reconstitution solvent are repeated several times. For example, in some embodiments, these steps are repeated 2, 3, 4, 5, 6, 7, 8, 9, or more times. In some embodiments, the two or more rounds of extractions are performed using different extractants and/or different extraction methods. For example, the fermentate may be subjected to both a solvent-based extraction and a chromatography-based extraction.

### Culture Medium

As used herein, a “culture medium” or “medium” is a composition designed to support the growth of a microbe. A culture medium may be a solid, liquid, or semi-solid composition. Typically, a culture medium comprises a carbon source (e.g., carbohydrates, alcohols), a nitrogen source (e.g., amino acids, ammonium salt), and micronutrients (e.g., metals, cofactors, vitamins). A medium may also comprise a salt used for osmolarity balance (e.g., sodium, chloride, potassium) and/or a pH buffering agent (e.g., a phosphate). However, the culture medium used with the present invention only needs to comprise a source of amino acids.

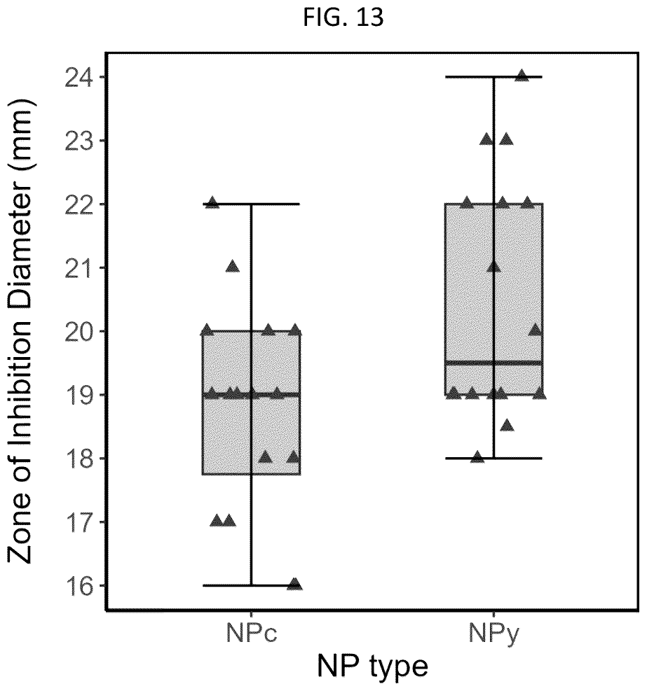

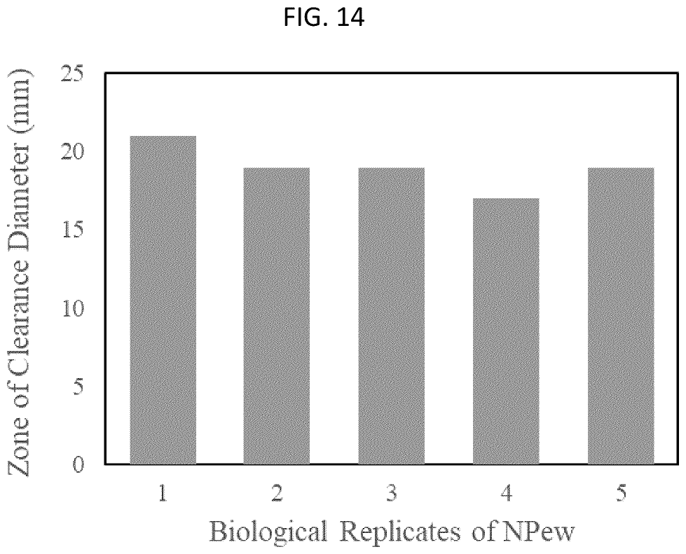

As used herein, the term “source of amino acids” refers to any material comprising amino acids. Examples of suitable sources of amino acids include proteins, peptides, peptones (i.e., soluble mixtures of polypeptides and amino acids that are formed by the partial hydrolysis of proteins), protein hydrolysates, and individual amino acids. The source of amino acids may be derived from an animal, plant, or fungus, or may be synthesized. In Example 4, the inventors demonstrate that fungal fermentates with antimicrobial activity can be produced by incubating A. oryzae in a variety of different amino acid sources, including yeast extract, egg white, whey protein, a plant-based protein mixture, soy protein isolate, essential amino acids, and hydrolyzed bone broth protein (see FIG. 13 and FIG. 14). Thus, in some embodiments, the source of amino acids is one of these particular food-grade ingredients. In some embodiments, the medium consists or consists essentially of only the source of amino acids and water.

In the Examples, the inventors extensively tested the antimicrobial activity of fermentates produced using a medium containing only yeast extract and water. This medium is referred to herein as “NPy medium” and the fermentates produced using this medium are referred to as “NPy”. Thus, in certain preferred embodiments, the source of amino acids is a yeast extract. “Yeast extract” is a composition comprising the cell contents of yeast without the cell walls. Typically, a yeast extract is created by heating yeast until the cells rupture and yeast enzymes begin to digest the cell contents.

In the Examples, the inventors also extensively tested the antimicrobial activity of fermentates produced using a medium containing only pancreatic digest of casein and water. This medium is referred to herein as “NPc medium” and the fermentates produced using this medium are referred to as “NPc”. However, the use of said medium was previously disclosed in International Patent Application Publication No. WO 2023/102065. Thus, the medium used in the methods of the present invention does not comprise casein or an enzymatic digest of casein. Casein is the main protein found in milk and cheese.

In Example 1, the inventors tested media comprising various concentrations of yeast extract, ranging from 3.2 to 36.8 g/L. They found that, within the tested range, the concentration of yeast extract did not significantly affect the antimicrobial activity of the fermentate. Thus, the culture medium may comprise the source of amino acids at a concentration of about 3-40 g/L, about 5-30 g/L, about 7-20 g/L, or about 9-12 g/L. However, the inventors discovered that using a lower concentration of substrate (i.e., 10 g/L) made it easier to filter the fermentate. Thus, in certain embodiments, the concentration of the source of amino acids is about 10 g/L. In Example 4, the inventors tested other sources of amino acids at a concentration of 20 g/L. Thus, in other embodiments, the concentration of the source of amino acids is about 20 g/L.

In preferred embodiments, the medium comprises only food-grade ingredients. As used herein, the term “food grade” is used to refer to substances that are non-toxic and safe for consumption by humans and/or animals.

### Antimicrobial Compositions

In a second aspect, the present invention provides antimicrobial compositions produced by the methods described herein. In some embodiments, the antimicrobial composition is food grade.

The term “antimicrobial” is used herein to describe a composition that kills or inhibits the growth or proliferation of a microorganism, such as a bacterium or fungus. Thus, the antimicrobial compositions may have antibacterial activity, antifungal activity, or both antibacterial activity and antifungal activity.

In some embodiments, the antimicrobial composition is an antibacterial composition. An “antibacterial composition” is a composition that kills or inhibits the growth or proliferation of a bacterium. Antibacterial compositions may have antibacterial activity against Gram-positive bacteria and/or Gram-negative bacteria. Antibacterial compositions may have bactericidal activity and/or bacteriostatic activity. The antibacterial activity of a composition can be assessed using an assay that measures the growth, proliferation, or viability of a bacterium in a sample following treatment with the composition. In Example 2, the inventors demonstrate that the A. oryzae fermentate NPy has antibacterial activity against Staphylococcus aureus, Escherichia coli, Klebsiella pneumoniae, Pseudomonas aeruginosa, Salmonella enterica, and Listeria monocytogenes. Thus, in some embodiments, the composition has antibacterial activity against one or more of these bacteria. In some embodiments, the composition has antibacterial activity against one or more bacterium selected from the specific strains/isolates of bacteria listed in Table 3. In Example 2, the inventors demonstrate that NPy has bactericidal activity. Thus, in some embodiments, the composition is bactericidal. Further, in Example 2, the inventors demonstrate that NPy cell-free extract has antibacterial activity against Acinetobacter baumannii, Cutibacterium acnes, Enterococcus faecium, and Listeria monocytogenes (see Table 8). Thus, in some embodiments, the composition has antibacterial activity against one or more of these bacteria.

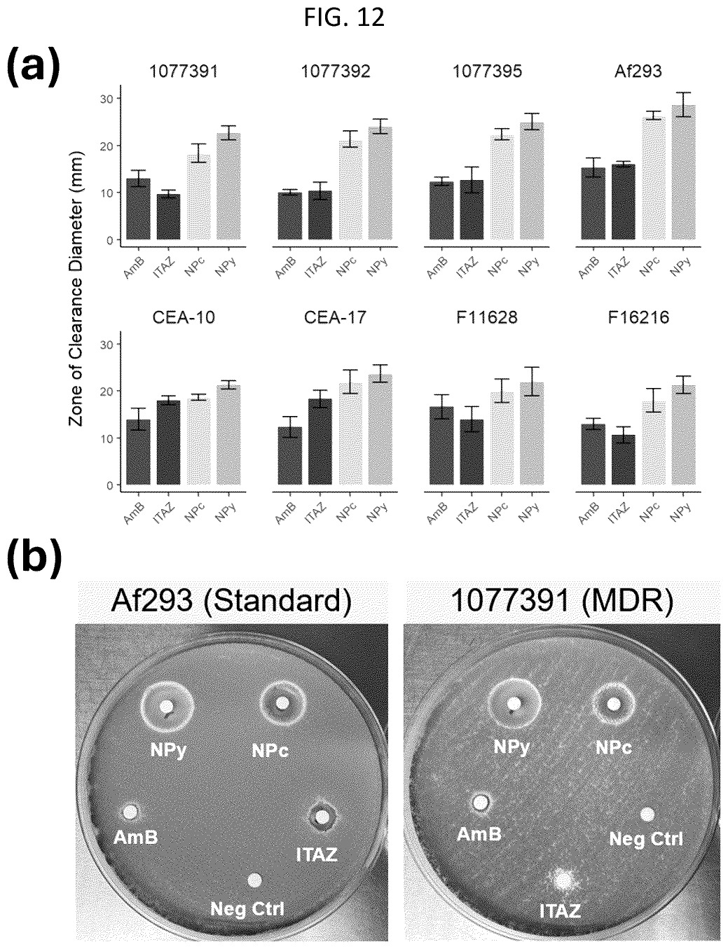

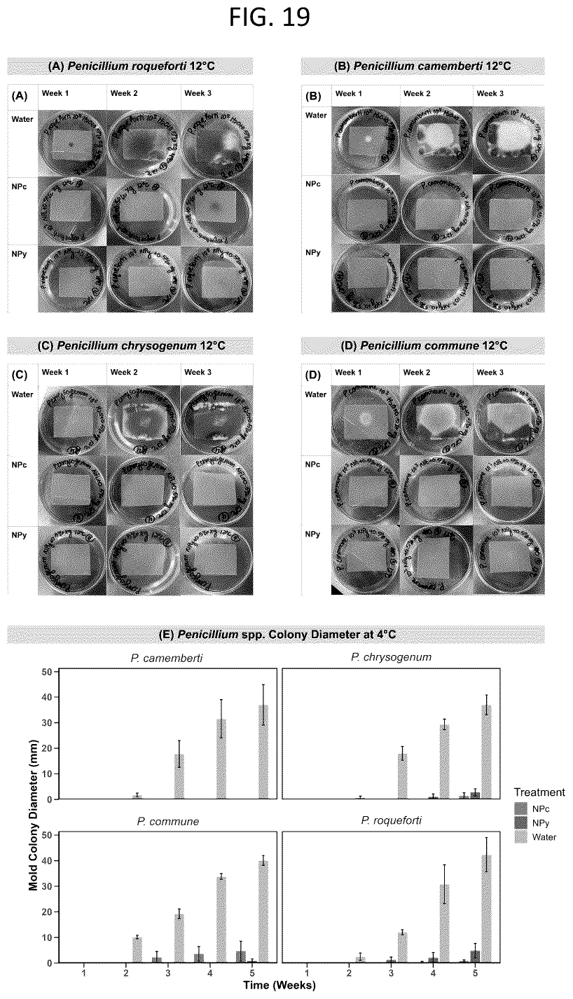

In some embodiments, the antimicrobial composition is an antifungal composition. An “antifungal composition” is a composition that kills or inhibits the growth or proliferation of a fungus. Antifungal compositions may have fungicidal activity and/or fungistatic activity. The antifungal activity of a composition can be assessed using an assay that measures the growth, proliferation, or viability of a fungus in a sample following treatment with the composition. In Example 2, the inventors demonstrate that NPy has antifungal activity against Candida albicans, Penicillium roqueforti, and Penicillium expansum. Thus, in some embodiments, the composition has antifungal activity against one or more of these fungi. In some embodiments, the composition has antifungal activity against the specific Candida albicans strain SC5314 and/or the specific Penicillium expansum strain PE-21. (Note: The Penicillium roqueforti used by the inventors was isolated from a cheese sample and was not given a strain number.) In Example 8, the inventors demonstrate that NPy has antifungal activity against Penicillium roqueforti, Penicillium camemberti, Penicillium chrysogenum, and Penicillium commune on cheese (see FIG. 19). Thus, in some embodiments, the composition has antifungal activity against one or more of these fungi. Further, in Example 2, the inventors demonstrate that NPy cell-free extract has antifungal activity against Aspergillus fumigatus (see FIG. 12) and Wickerhamomyces anomalus, Kluyveromyces marxianus, Pichia kudriavzevii, Candida lusitaniae, Clavispora lusitaniae, and Debaryomyces hansenii (see Table 8). Thus, in some embodiments, the composition has antifungal activity against one or more of these fungi.

In Example 6, the inventors demonstrate that the antimicrobial activity of the fermentates NPy and NPew (i.e., a fermentate produced by culturing A. oryzae in egg white protein) are heat stable. Specifically, they demonstrate that these fermentates retain antimicrobial activity after being heated to a temperature of at least 121° C. Thus, in some embodiments, the antimicrobial activity of the antimicrobial composition is heat stable.

In some embodiments, the antimicrobial composition comprises one or more additives. Appropriate additives include preservatives, chelating agents, antioxidants, cryoprotective agents, stabilizers, emulsifiers, texturizers, and gelling agents. Examples of preservatives include ethylenediaminetetraacetic acid (EDTA), parabens, sodium benzoate, and sorbic acid. Examples of chelating agents include nitrilotriacetic acid, EDTA, diethylene triamine penta-acetic acid (DTPA), propylene diamine tetra-acetic acid, and ethylene diamine-N,N″-di(hydroxyphenyl or hydroxy-methylphenyl) acetic acid. Examples of antioxidants include ascorbic acid, butylated hydroxy anisole, butylated hydroxyl toluene, methionine, sodium metabisulfite, phosphoric acid, tartaric acid, and tocopherol. Examples of cryoprotectants include gelatin, glycerol, milk, mannitol, and skim milk. Examples of stabilizers and texturizers include stearic acid, palmitic acid, stearyl alcohol, cetyl alcohol, behenyl alcohol, and polyethylene glycols. Examples of emulsifiers include cetyl trimethylammonium bromide, polyvinyl alcohol, and polyvinyl pyrrolidone. Examples of gelling agents include gelatin, gums, flax, and starches, such as xanthan gum, galactomannan gum, cassia starch, and pectin.

In some embodiments, the antimicrobial compositions further comprise a food-grade binding agent. As used herein, a “food-grade binding agent” is a non-toxic substance that holds the antimicrobial composition on the surface of a food. Examples of food-grade binding agents include waxes, resins, and gelling agents. In some embodiments, the food-grade binding agent is xanthan gum.

In some embodiments, the antimicrobial composition is formulated for oral administration. For example, it may be formulated as a tablet, a capsule, a powder, a troche, a syrup, a liquid suspension, an emulsion, a solution, or a beverage.

In some embodiments, the composition is formulated as a food additive. As used herein, the term “food additive” refers to a substance that is added to food to preserve it. In these embodiments, the compositions may include an edible carrier. Non-limiting examples of edible carriers include cornstarch, lactose, sucrose, bean flake, peanut oil, olive oil, sesame oil, and propylene glycol.

In other embodiments, the composition is formulated for topical administration. For example, it may be formulated as a shampoo, conditioner, mask, spray, paste, cream, ointment, lotion, oil, gel, tincture, powder, patch, or bandage.

### Products Treated With Antimicrobial Compositions

The antimicrobial compositions of the present invention were designed to inhibit the growth of microbes on and in various products and organisms. Thus, in a third aspect, the present invention provides products comprising the antimicrobial compositions described herein.

In the Examples, the inventors demonstrate that their antimicrobial compositions have activity against several foodborne pathogens. Thus, in some embodiments, the product is a food product. As used herein, a “food product” is a product that is prepared for consumption by a human or animal. Non-limiting examples of food products include grains, breads, nuts, seeds, fruits, meat products, dairy products, candies, cookies, pizza, noodles, gums, soups, beverages, and animal feeds. In specific embodiments, the food product is milk, yogurt, or cheese. The antimicrobial compositions may be included in the food product as a food additive that is mixed into all or a portion of the food product or may be applied to the surface of the food product. Additional ingredients may be added along with the antimicrobial composition to improve the taste of the resulting product. In some embodiments, the food product is processed using heat (e.g., for cooking, pasteurization, sterilization, pelletization). Because the antimicrobial activity of the antimicrobial compositions is heat stable, they may be applied to the food product either before or after it has undergone thermal processing.

In the Examples, the inventors demonstrate that their antimicrobial compositions have activity against several ESKAPE pathogens (i.e., antibiotic-resistant bacteria that are a global threat to clinical health) and human opportunistic fungal pathogens. Thus, in other embodiments, the product is a medical device. A “medical device” is any device intended to be used for a medical purpose. Examples of medical devices include instruments, apparatuses, implements, machines, appliances, implants, and in vitro reagents. In some embodiments, the medical device is a wound dressing.

### Methods of Use

In a fourth aspect, the present invention provides methods of killing or inhibiting the growth or proliferation of a microbe. The methods may be used to kill or inhibit the growth of microbes that are already present or may be used prophylactically (i.e., to prevent the growth of a microbe that was not yet present at the time the antimicrobial composition was applied or administered).

Growth inhibition may be measured as a reduction in the growth of the microbe in the presence of the antimicrobial compositions relative to growth of a control microbe. As used herein, a “control microbe” is a comparable microbe (e.g., of the same species, strain, isolate) grown under the same or comparable conditions but was grown in the absence of the antimicrobial composition. Inhibition of microbe growth or proliferation can be detected using known growth inhibition assays, including zone of inhibition assays, assays that utilize the concentration, number, or optical density (e.g., absorbance at a particular wavelength) of microbes as a readout of microbial growth, and assays that detect the presence of a biomarker (e.g., aflatoxin B1) as an indicator of microbial growth. Such assays may be automated using a growth curve analysis system, such as the Bioscreen C.° system. Microbe killing can be detected using cell viability assays such as tetrazolium reduction assays (e.g., MTT, MTS, XTT, and WST-1), resazurin reduction assays (alamarBlue®), real-time cell viability assays (e.g., RealTime-Glo™ MT Cell Viability Assay from Promega), and assays that measure protease activity or ATP within cells as an indicator of viability.

In the present methods, the antimicrobial composition should be applied or administered in an “effective amount,” i.e., an amount sufficient to kill the microbe or to reduce, inhibit, or prevent the growth or proliferation of the microbe. An effective amount of an antimicrobial composition can be estimated initially in vitro, in cell culture assays, or in an animal model. For example, a minimum inhibitory concentration (MIC), i.e., the lowest concentration of an antimicrobial agent that inhibits the growth of a particular microbe, can be determined using a MIC assay and used as the minimal effective amount. Alternatively, a minimum bactericidal concentration (MBC) test can be used to determine the lowest amount of an antimicrobial agent that results in microbial death.

In some embodiments, the methods are used to inhibit the growth of a microbe in or on a food or feed product. These methods comprise applying an effective amount of an antimicrobial composition described herein to the food or feed product. In these embodiments, the antimicrobial composition is preferably food grade.

In other embodiments, the methods are used to inhibit the growth of a microbe on a surface. These methods comprise applying an effective amount of an antimicrobial composition described herein to the surface. Examples of surfaces that can be treated using the present methods include, without limitation, the surface of a medical device, the surface of a desk or bench, the surface of a food package, a food preparation surface (e.g., a surface used for cooking or food manufacturing), the surface of a plant, and the skin, hair or fur of a human or animal.

The antimicrobial compositions may be applied to a product in several ways. For example, a product may be dipped in, coated with, or sprayed with the antimicrobial composition. In these cases, the antimicrobial composition may comprise a binding agent (e.g., xanthan gum, wax coating) to improve its ability to stick to the surface of the product. Alternatively, the antimicrobial composition may be impregnated or mixed into all or a portion of the product. The antimicrobial compositions may be applied to the product in a liquid form or may be dried and/or concentrated and applied to the product in a powdered form. In some embodiments, the antimicrobial composition is provided in the form of an antimicrobial wipe.

In still other embodiments, the methods are used to inhibit the growth of a microbe in or on a subject. These methods comprise administering an effective amount of an antimicrobial composition described herein to the subject. In these embodiments, the methods may be used to treat or prevent a microbial infection. Examples of microbial infections include, without limitation, food poisoning, whooping cough, strep throat, ear infection, urinary tract infection, thrush, vaginitis, candidiasis, ringworm, and athlete's foot. The methods may also be used to treat or prevent conditions in which microbes play a significant role, such as acne, dandruff, and dermatitis. When using the compositions for treating skin conditions the mechanism of action for the compositions may not be limited to antimicrobial effects and the compositions may have other modes of action to yield improvements to skin texture and tone and may show anti-aging effects as well. The compositions provided in International Patent Application Publication No. WO 2023/102065 may also be used in these methods and the compositions are incorporated herein by reference.

The inventors have preliminary data that suggests that their antimicrobial compositions can be used for hair restoration, i.e., to increase hair growth and/or decrease hair loss. These data suggest that applying a shampoo containing 25% NP fermentate stimulates new hair growth and makes hair follicles stronger. Thus, in other embodiments, the methods simply comprise administering an antimicrobial composition described herein to a subject. In preferred embodiments, the administration is topical. The compositions may be applied to hair or skin, for example. In some embodiments, the antimicrobial composition is administered as part of a hair product. Examples of suitable hair products include, but are not limited to, shampoo, conditioner, serum, gel, pomade, clay, paste, mousse, spray, oil, mask, and cream. Notably, the mechanism by which NP fermentate affects hair is unclear, and the inventors suspect that it does not depend on the antimicrobial activity of the fermentate. Thus, in these embodiments, the antimicrobial composition may have a desirable effect on hair via a mechanism other than antimicrobial activity. The inventors wish not to be limited by any mechanism of action for use of the compositions comprising an Aspergillus oryzae fermentate for application to skin or hair of a subject. When using the compositions for hair applications, the mechanism of action for the compositions may not be limited to antimicrobial effects and the compositions may have other modes of action to yield improvements to hair growth, texture and tone that are not due to antimicrobial activity of the compositions. The compositions provided in International Patent Application Publication No. WO 2023/102065 may also be used in these methods and the compositions are incorporated herein by reference.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is human.

As used herein, the term “administering” refers to the introduction of a substance into or onto a subject's body. Methods of administration are well known in the art and include, but are not limited to, oral administration, transdermal administration, administration by inhalation, nasal administration, topical administration, intravaginal administration, ophthalmic administration, intra-aural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

The present invention is based on the inventors' creation of cell-free fermentates with antimicrobial activities. The fermentates are produced by culturing Aspergillus oryzae in various food-safe media and then filtering out the fungal cells. These fermentates are referred to herein as “NP,” short for “natural preservative.” Specifically, fermentate produced by culturing A. oryzae in a medium comprising pancreatic digest of casein is referred to as “NPc,” fermentate produced by culturing A. oryzae in a medium comprising yeast extract is referred to as “NPy,” and fermentate produced by culturing A. oryzae in a medium comprising dried egg white protein is referred to as “NPew.” In the following Examples, the inventors demonstrate that their fermentates exhibit potent antimicrobial activities against both bacteria and fungi.

### Example 1: Development of NPy

### Identification of the Essential Component of NP1 Medium

The details of the preparation of NP1 fungal fermentates were previously described in International Patent Application Publication No. WO2023/102065. To identify the essential component of NP1 media (i.e., the component required for the antimicrobial activity of NP1 fermentate), we prepared fermentates by growing Aspergillus oryzae in various modified NP1 culture broths lacking one component of the NP1 medium (i.e., NP1 without maltose, NP1 without yeast extract) or comprising only a single ingredient of the NP1 medium (i.e., 6 g/L malt extract only, 1.8 g/L maltose only, 6 g/L glucose only, 1.2 g/L yeast extract only (original concentration), 4.8 g/L yeast extract only (elevated concentration)). After harvesting, each fermentate was mixed with 24 g/L TSB powder and sterilized via autoclave (15 psig and 121° C. for 20 minutes). The antimicrobial activities of the fermentates against different pathogens were then tested in a growth curve experiment.

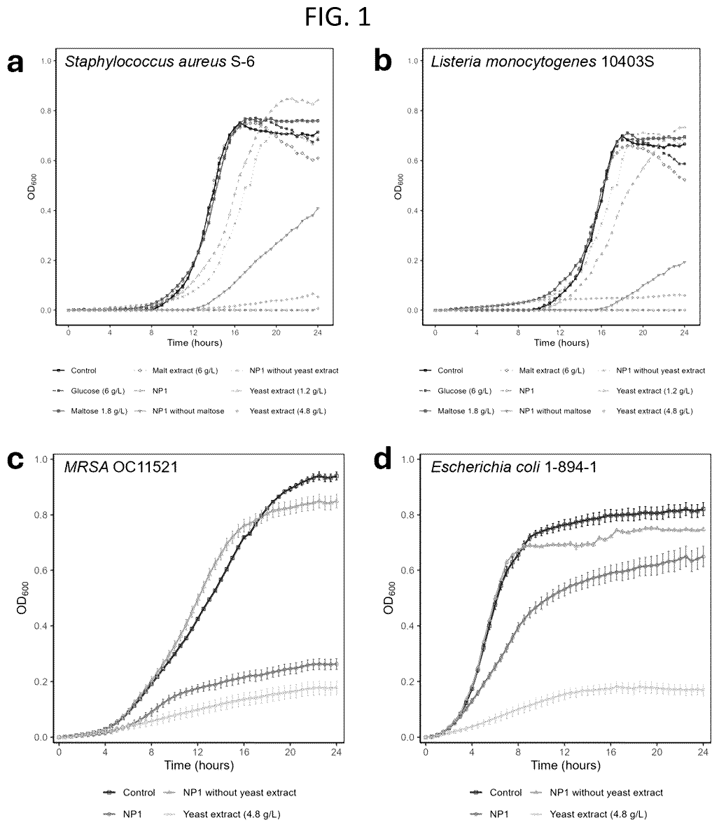

Results: The medium formulated with heat-treated NP1 fermentate devoid of yeast extract permits the proliferation of S. aureus and L. monocytogenes (FIG. 1). In contrast, the presence of yeast extract in the fermentate at concentrations exceeding 4.8 g/L effectively inhibits all bacterial growth including MRSA and E. coli (FIG. 1). Thus, yeast extract is both a necessary and sufficient component for conferring antimicrobial activity to NP1 fungal fermentates.

### Determination of the Fermentation Parameters of NPy

For the initial characterization of “NPy” (i.e., fermentate prepared in a medium comprising only yeast extract), the fermentation parameters were arbitrarily set to the levels that were found to be optimal for NPc (20 g/L substrate level, 10⁵ conidia/mL inoculation level, 6 days of incubation time at 30° C. and 220 rpm agitation). However, we recognized that these fermentation parameters may not be optimal (i.e., they may not produce the greatest antimicrobial activity) for NPy. Therefore, the impact of fermentation parameters on the antimicrobial activity of NPy was evaluated. Based on the preliminary data produced using the NPc fermentation parameters, a response surface methodology (RSM) using a rotatable central composite design (RCCD) was established, which aimed to optimize the following three parameters: concentration of substrate (i.e., yeast extract), incubation time, and amount of A. oryzae NRRL 3483 spores inoculated. The response being measured in this experiment is the diameter of the clearance zone. The tested levels of each of the three investigated fermentation parameters are detailed in Table 1, and based on these levels, a RSM RCCD table was generated using JMP Pro 17 software as shown in Table 2. Each run indicated in Table 2 represents an individual NPy product that was derived from 150 mL of fermentate fermented using the parameters in its corresponding row. All runs were fermented at 30° C. at a shaking speed of 220 rpm. The NPy from each run was harvested after its corresponding incubation time and filtered through Whatman Grade GF/A glass microfiber filter. Then, the antimicrobial activity of each NPy filtrate was assessed against S. aureus S-6 by disk diffusion assay. The diameter of the zone of clearance was measured for each run as the response in RSM design. The experiment was 5 done in biological triplicates, and the runs in the first replicate were randomized using JMP Pro 17, as shown in Table 2, and the following two biological replicates had their runs re-randomized by R 4.3.1. For each run, the average diameter from three biological replicates was input into the JMP Pro 17 design table to run the RSM model and evaluate the potential impact of the three investigated fermentation parameters on the antimicrobial potency of NPy 10 fermentates.

TABLE 1
Tested fermentation parameter levels

| Fermentation | Axial | Minimum | Center | Maximum | Axial |
|---|---|---|---|---|---|
| parameters | (−α) | (−1) | (0) | (+1) | (+α) |
| Incubation time | 1 | 3 | 6 | 9 | 11 |
| (days) | | | | | |
| Inoculation level | 3.3 | 4.0 | 5.0 | 6.0 | 6.7 |
| (log conidia/mL) | | | | | |
| Substrate concentration | 3.2 | 10.0 | 20.0 | 30.0 | 36.8 |
| (g/L) | | | | | |

TABLE 2
Input for the response surface methodology (RSM) model,
detailing the fermentation parameters used in each run

| | Incubation | Inoculation | Substrate |
|---|---|---|---|
| | time | level (log | concentration |
| Run^{†} | (days) | conidia/mL) | (g/L) |
| 1 | 9 | 4.0 | 30.0 |
| 2 | 1 | 5.0 | 20.0 |
| 3 | 6 | 5.0 | 20.0 |
| 4 | 6 | 6.7 | 20.0 |
| 5 | 6 | 5.0 | 36.8 |
| 6 | 6 | 3.3 | 20.0 |
| 7 | 3 | 6.0 | 30.0 |
| 8 | 3 | 6.0 | 10.0 |
| 9 | 11 | 5.0 | 20.0 |
| 10 | 9 | 6.0 | 10.0 |
| 11 | 6 | 5.0 | 20.0 |
| 12 | 9 | 6.0 | 30.0 |
| 13 | 9 | 4.0 | 10.0 |
| 14 | 6 | 5.0 | 20.0 |
| 15 | 3 | 4.0 | 10.0 |
| 16 | 6 | 5.0 | 20.0 |
| 17 | 6 | 5.0 | 3.2 |
| 18 | 6 | 5.0 | 20.0 |
| 19 | 3 | 4.0 | 30.0 |

^{†}The order of runs was re-randomized for the second and third biological replicates.

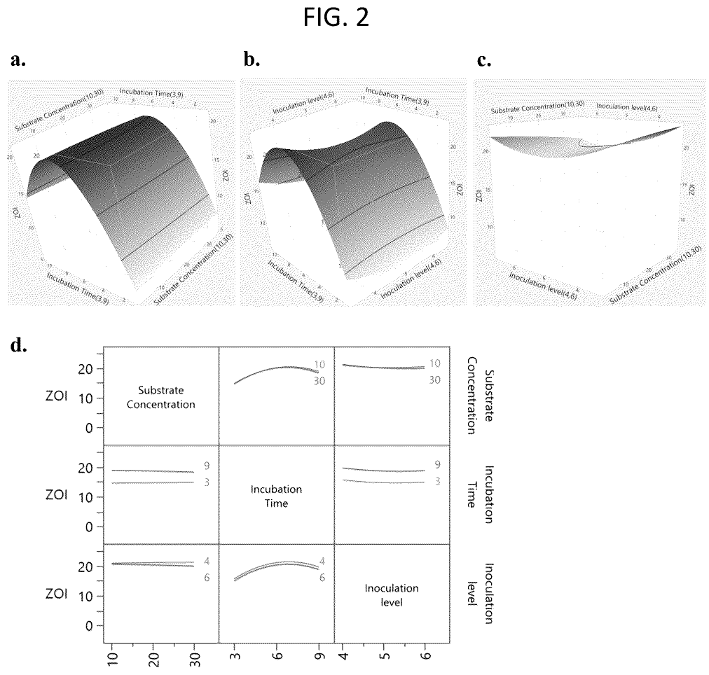

Results: Response surface models were generated based on the RSM experiments (FIG. 2). Within the investigated ranges of fermentation parameters, the values that maximize the antimicrobial strength of NPy were identified. For incubation time, the best value was determined to be 6.78 days; for inoculation level, the best value was determined to be 4 log conidia/mL; and for substrate concentration, the best value was determined to be 30 g/L yeast extract. Incubation time was determined to be the only fermentation parameter that affected the antimicrobial potency of the final NPy fermentate in a statistically significant manner (p=0.00047). In contrast, inoculation level had a p-value of 0.43, and substrate concentration had a p-value of 0.87, suggesting that these two fermentation parameters, within the ranges investigated by the RSM experiments, do not significantly impact the antimicrobial strength of NPy. Strong antimicrobial activity against S. aureus S-6 (i.e., a zone of inhibition diameter of 18.3 mm) was observed with even the lowest substrate concentration (i.e., 3.2 g/L). Therefore, the substrate concentration was arbitrarily set to 10 g/L for further experiments because use of a less concentrated substrate facilitates the filtration of the post-fermentation product. The incubation time was rounded down to 6 days for further experiments, which did not significantly influence the antimicrobial strength of NPy according to the prediction from the established RSM model.

Thus, for fermentation of NPy, the substrate concentration was set at 10 g/L yeast extract; the inoculation level of A. oryzae NRRL 3483 spores was set at 10⁴ conidia/mL; and the incubation time was set at 6 days of incubation at 30° C. with 220 rpm agitation. Unless otherwise noted, NPy was produced using these parameters. According to the RSM model, NPy fermentate produced by using these fermentation parameters was expected to generate a zone of clearance with a diameter of 21.3 mm against S. aureus S-6 when 100 μL of 20× extract was loaded on a 6-mm antimicrobial susceptibility disk. This predicted datum was later found to be consistent with the average zone of clearance obtained from 16 batches of NPy produced using the adjusted fermentation parameters size (20.47 mm with a standard deviation of 1.92 mm, as mentioned in Example 2).

### Example 2: Antimicrobial Activities of NPy and NPc

### Antimicrobial Effects of NPy

The antimicrobial effects of NPy fermentates were screened against a wide array of pathogenic microbial strains using a growth curve experiment. NPy fermentate derived from A. oryzae NRRL 3483 was used in this experiment. RO water, NPy, and 50% v/v NPy solution (NPy:RO water=1:1) were mixed with 30 g/L of TSB as a nutrient support for bacterial growth and then sterilized by a liquid autoclave cycle (15 psig for 20 minutes at 121° C.). Then, for each concentration of NPy in TSB media, 180 μL of the media were loaded into three wells of each column of a 100-well lidded honeycomb microplate (Bioscreen and Growth Curves Ltd., Telekatu 12, 20360 Turku, Finland).

In total, 20 bacterial isolates alongside one isolate of the opportunistic pathogenic yeast Candida albicans were screened by the growth curve experiments to evaluate the antimicrobial effectiveness of NPy. The tested strains are listed in Table 3. Bacteria were resuscitated on TSA from a cryotube glycerol stock stored at −80° C. Then, a culture of each bacterium was prepared in a TSB solution with adjustment to an OD₆₀₀ value between 0.08 and 0.1. Then, 20 μL of the adjusted bacterial cultures were loaded into each well in the corresponding column of the 100-well plate. The inoculated 100-well plates were loaded into the Bioscreen C.° Pro machine (Bioscreen and Growth Curves Ltd.) to track the growth of bacteria at 37° C. by measuring OD₆₀₀ every 30 minutes for 24 hours.

TABLE 3
Pathogenic microbes screened for NPy activity

| Species | Strain/Isolates |
|---|---|
| Staphylococcus aureus | OC 4222, isolate 56/24, methicillin-resistant |
| | OC 4222, isolate 56/56, methicillin-resistant |
| | OC 17042, isolate 56/56, methicillin-resistant |
| | OC 04-045, isolate 56/56, methicillin-resistant |
| | OC 8530, isolate 56/25, methicillin-resistant |
| | OC 8530 isolate 56/26, methicillin-resistant |
| | OC 8530, isolate 56/60, methicillin-resistant |
| | OC 11521, isolate 56/57, methicillin-resistant |
| | MW2, methicillin-resistant |
| | S-6 |
| | ATCC 33593 |
| Escherichia coli | 1-894-1 |
| | 2671 |
| Klebsiella pneumoniae | 81-1269A |
| | BAA-2146 |
| Pseudomonas aeruginosa | 65 |
| | 2638 |
| | 3060 |
| Salmonella enterica | serovar Typhimurium strain S9 |
| Listeria monocytogenes | 10403S |
| Candida albicans | SC5314 |

NPy was fermented as described in Example 1 and filtered through Whatman Grade GF/A glass microfiber filter. Solutions of 0, 50, and 100% v/v NPy were used as the solvent to reconstitute TSB powder at 30 g/L to make the substrates for bacterial growth. The compositions of these substrate solutions are detailed in Table 4. For each concentration of NPy, the corresponding medium substrate was pipetted into three wells in a Bioscreen 100-well plate, with each well containing 180 μL of the substrate. The aforementioned bacterial cultures were then prepared and pipetted into each corresponding well with aliquots of 20 μL. Then the inoculated 100-well plates were loaded into the Bioscreen C.° Pro machine to track the growth of bacteria by measuring OD₆₀₀ every 30 minutes for a 24-hour incubation at 37° C.

TABLE 4
Substrate compositions for bacterial growth curve experiments

| Treatment | Composition of the substrate |
|---|---|
| 0% NPy | 100 mL RO water + 3 g tryptic soy broth powder |
| 50% NPy | 50 mL RO water + 50 mL NPy + 3 g tryptic soy broth powder |
| 100% NPy | 100 mL NPy + 3 g tryptic soy broth powder |

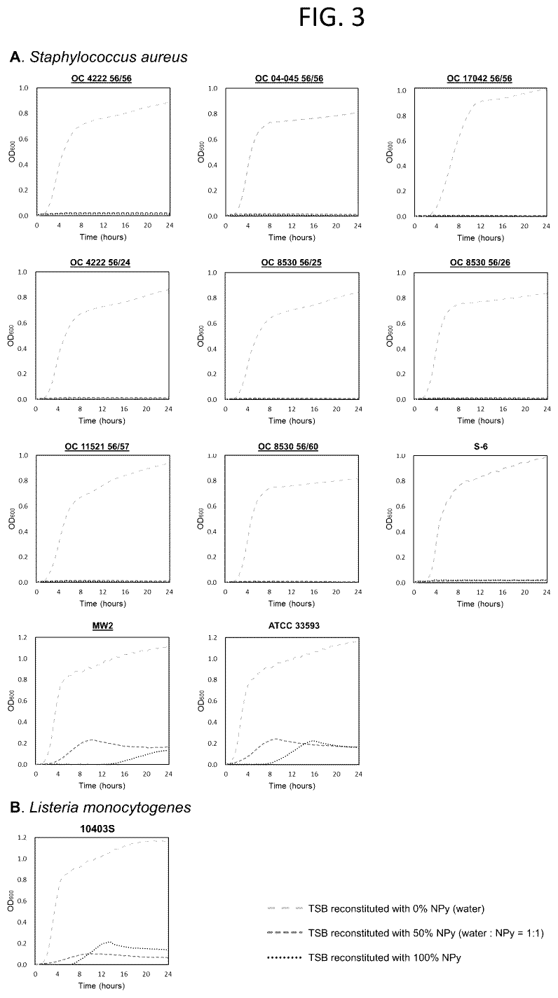

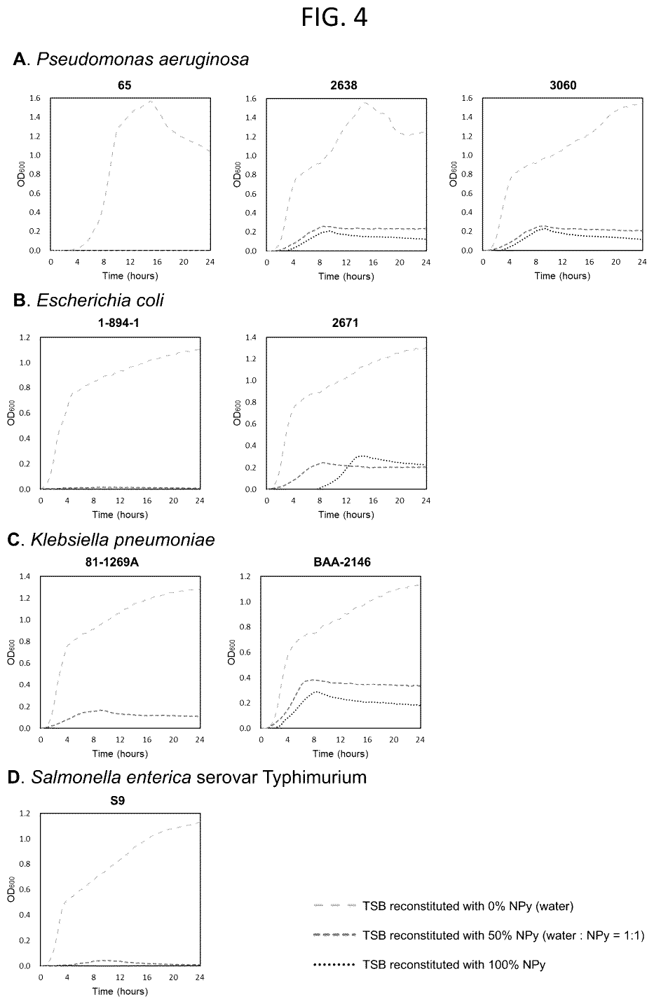

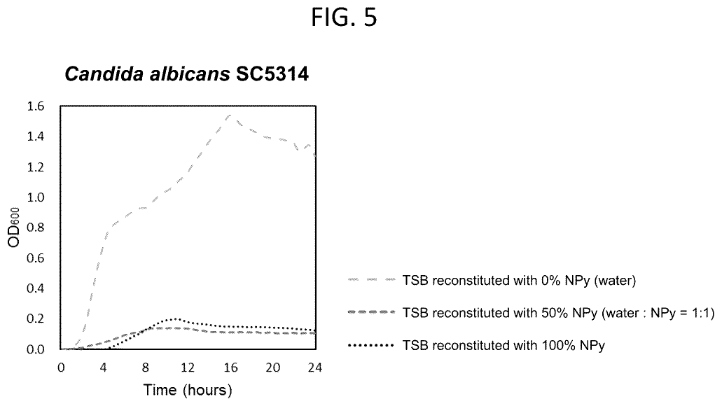

Results: All investigated bacteria, including both Gram-positive (FIG. 3) and Gram-negative (FIG. 4) pathogens, were inhibited by the addition of NPy. Many bacteria did not grow at all when NPy was supplied at 50% and 100%, whereas some strains had delayed growth with an increase of OD₆₀₀ of less than 0.3. Compared to the bacterial growth in TSB without NPy addition, these delays in growth were significant, as the bacteria reached stationary phase at a much lower population density in the broth. C. albicans could not grow when the growth medium was reconstituted with 50% or 100% NPy solution (FIG. 5).

### Bactericidal Effects of NPy

To assess whether NPy kills pathogenic bacteria or simply inhibits their growth, the plate-count method was used to enumerate the concentration of live bacteria during NPy treatments. Escherichia coli FRIK 47 was used for assessing the bactericidal effects of NPy.

Mueller Hinton Broth (MHB, which contains beef infusion solids at 2 g/L, starch at 1.5 g/L, and casein hydrolysate at 17.5 g/L) powder was added to NPy at 21 g/L to supply the nutrients necessary for the growth of the aforementioned bacterium. MHB media, reconstituted with NPy and RO water, were sterilized by a liquid autoclave cycle (15 psig for 15 minutes at 121° C.).

Bacteria were resuscitated from glycerol cryotubes stored at −80° C. onto a TSA plate, which was then incubated at 37° C. overnight. Grown colonies of bacteria were picked and transferred into tubes containing 10 mL of sterilized MHB, which were then incubated at 37° C. for the bacteria to grow. The OD₆₀₀ of the bacterial cultures was measured and adjusted to 0.08-0.1. The target inoculation level was 5×10⁵ CFU/mL, and based on the actual OD₆₀₀ value, the volume of adjusted bacteria culture was calculated and delivered into 10 mL of the prepared media of MHB reconstituted with RO water and with NPy. The inoculated cultures were vortexed and then incubated at 37° C. At each sampling point, 100 μL of the bacterial cultures were sampled and serially diluted with phosphate-buffered saline (PBS; which contains NaCl at 8 g/L, KCl at 0.2 g/L, Na₂HPO₄ at 1.44 g/L, and KH₂PO₄ at 0.24 g/L). Then, 100 μL of proper dilutions were spread-plated on TSA, and the plates were incubated at 37° C. overnight. The number of bacterial colonies were enumerated after incubation.

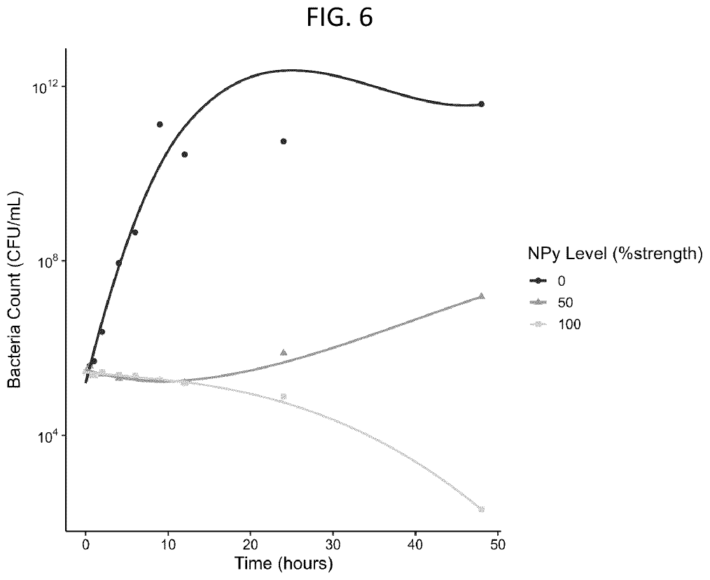

Results: A bactericidal effect was observed for NPy. Within 48 hours post-inoculation, a 3-log decrease was observed for bacterial density of E. coli FRIK 47 in NPy-reconstituted MHB media (FIG. 6).

### Antifungal Effects of NPy

The antifungal effect of NPy was evaluated by spot-plating fungal spores on agar plates that contained different concentrations of NPy fermentate. Agar media were made with PDA powder solubilized by different percentages of NPy in RO water. The compositions of the various agar media are detailed in Table 5. The agar media were sterilized by a liquid autoclave cycle (15 psig for 20 minutes at 121° C.) and then poured into 150-mm diameter petri-dishes. Penicillium roqueforti and Penicillium expansum PE-21 were used in this experiment. A spore suspension of each strain was made as described below, in the section titled “Evaluation of the antifungal effects of NPc.” The concentration of spores was quantified by counting using a hemacytometer under the microscope. Spore suspension was then diluted in PBS to the concentrations of 10³, 10⁴, 10⁵, and 10⁶ conidia/mL as the inocula. Then, on agar plates made with different concentrations of NPy in the media, 10 μL of each inoculum were spot-plated on the agar plate. For a specific strain, the inoculum containing four different concentrations of spores in PBS was spot-plated side by side, along with a negative control of a 10 μL PBS. The agar plates were incubated at room temperature (˜25° C.) for five days. Photos were taken after the incubation period.

TABLE 5
Compositions of the agar media used to
test the antifungal activity of NPy

| Treatment | Composition of the agar medium |
|---|---|
| 0% NPy | 200 mL RO water + 7.8 g potato dextrose agar powder |
| 10% NPy | 180 mL RO water + 20 mL NPy + 7.8 g potato dextrose |
| | agar powder |
| 25% NPy | 150 mL RO water + 50 mL NPy + 7.8 g potato dextrose |
| | agar powder |
| 50% NPy | 100 mL RO water + 100 mL NPy + 7.8 g potato dextrose |
| | agar powder |
| 75% NPy | 50 mL RO water + 150 mL NPy + 7.8 g potato dextrose |
| | agar powder |
| 100% NPy | 200 mL NPy + 7.8 g potato dextrose agar powder |

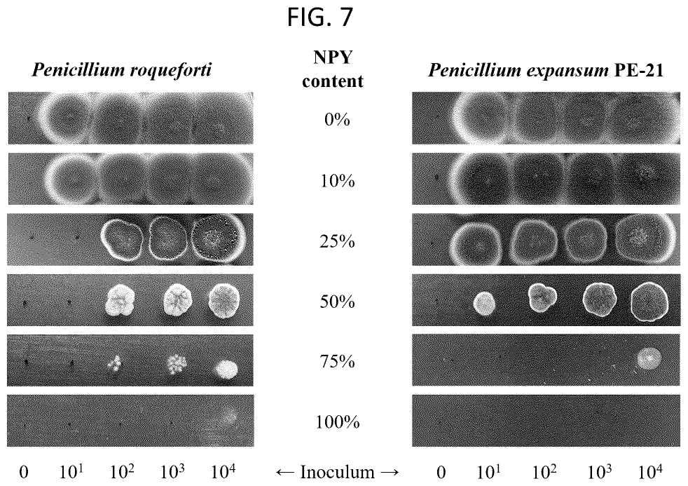

Results: The colony size of both P. expansum PE-21 and P. roqueforti were constrained when 25% NPy was used to make the agar media (FIG. 7). Conidiation was mostly inhibited at a concentration of NPy at 75%, and almost no growth was observed for both species when the PDA medium was reconstituted 100% with NPy fermentate.

### Bactericidal Effects of NPc

The previously described fermentate NPc was further characterized along with NPy to understand its antimicrobial actions. Specifically, the antibacterial effect of NPc was tested against S. aureus, L. monocytogenes, E. coli, S. Typhimurium, K. pneumoniae, and P. aeruginosa. TSB was reconstituted in NPc fermentate as a nutrient supplement for bacterial growth. A control of TSB reconstituted with water was included for each bacterium. Given the knowledge that Gram-negative bacteria appear to be more resistant to NPc, a negative control using PBS and a sample of NPc fermentate without TSB supplementation were also included in the experiment. Fresh bacterial cultures were inoculated into each of the substrates to a level of 10⁵ per mL. The number of bacteria in the culture were determined immediately after inoculation and after 18 hours of incubation at 37° C. by the plate-count method. In addition, the bactericidal effect of NPc was also assessed against enterohemorrhagic E. coli (EHEC) O157:H7 strain FRIK 47, Salmonella enterica serovar Typhimurium S9, and methicillin-resistant S. aureus OC 11521 by growing these bacteria in NPc-reconstituted MHB and measuring bacterial growth at several timepoints.

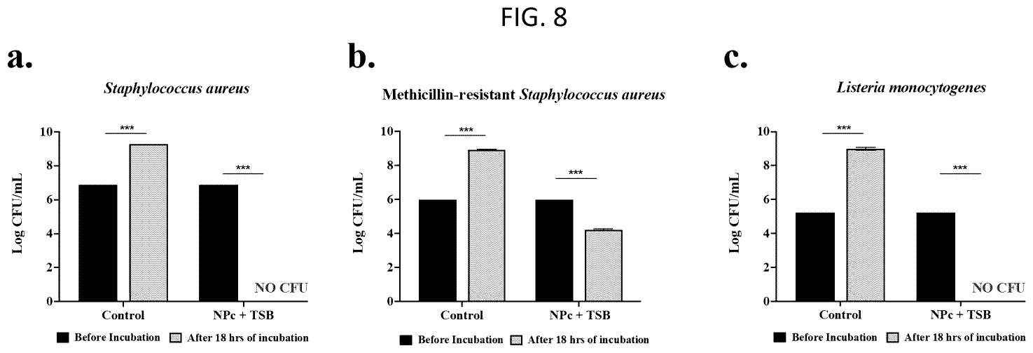

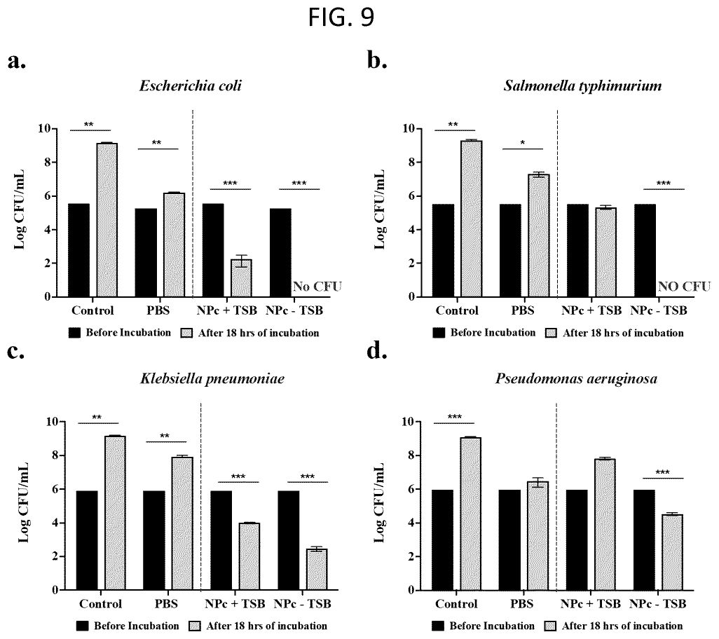

Results: After growing S. aureus (FIG. 8a) and L. monocytogenes (FIG. 8c) in TSB liquid medium prepared with NPc for 18 hours, no cells were detected, indicating a bactericidal effect. MRSA showed a reduction of 1.5 logarithmic units (FIG. 8b). For gram-negative bacteria, after growing E. coli (FIG. 9a) and S. Typhimurium (FIG. 9b) in TSB liquid medium prepared with NPc for 18 hours, there were 3.4 log (equivalent to a >1000-fold reduction) and no reductions observed, respectively. However, when grown in NPc without added nutrients, no cells of E. coli and S. Typhimurium were detected. The same outcome was noted with K. pneumoniae and P. aeruginosa. In the case of K. pneumoniae (FIG. 9c), TSB liquid medium prepared with NPc showed a 2-log reduction (equivalent to a 100-fold reduction), while NPc without added nutrition showed a greater reduction of 3.5 log (equivalent to a >1000-fold reduction).

No inhibition of P. aeruginosa was detected in TSB liquid medium prepared with NPc, whereas a 1.5 log reduction (equivalent to a >10-fold reduction) was noted in NPc without added nutrition (FIG. 9d). Due to its biofilm formation, achieving consistent data was challenging for P. aeruginosa. NPc can serve as a viable alternative solution for effectively controlling the growth of ESKAPE pathogens.

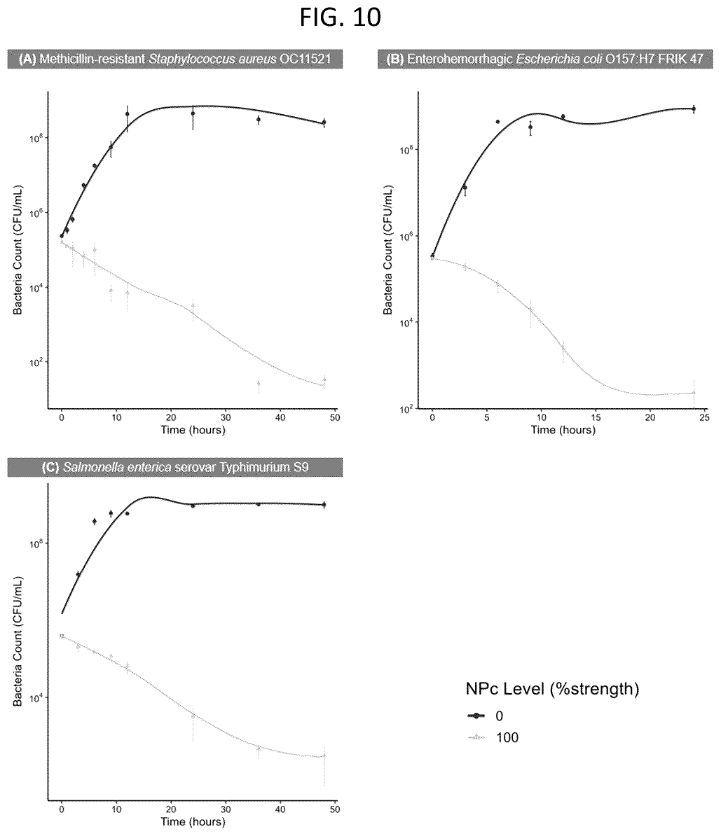

The bactericidal effect of NPc was also assessed against MRSA OC 11521, enterohemorrhagic E. coli strain FRIK 47, and S. Typhimurium S9 (FIG. 10). All three strains were killed when NPc, instead of water, was used to reconstitute the media powder. A 3-log reduction was achieved within 24 hours post-inoculation for E. coli FRIK 47, and within 48 hours post-inoculation for MRSA OC 11521 and S. Typhimurium S9.

### Antifungal Effects of NPc

The antifungal effects of NPc were evaluated in a broth condition. To prepare NPc-containing broths for the antifungal tests, NPc was first diluted with RO water to different percentages, and for each concentration, the solution was mixed with 24 g/L PDB powder, followed by a liquid autoclave cycle to sterilize the media (15 psig for 20 minutes at 121° C.). The compositions of the media are detailed in Table 6. The antifungal activity was tested against four strains of Aspergillus fumigatus and four species of Penicillium, which are listed in Table 7. A. fumigatus strains and Penicillium spp. from the glycerol cryotubes stored at −80° C. were reactivated onto PDA plates. The plates of A. fumigatus strains were incubated at 37° C. for three days, and the plates of Penicillium spp. were incubated at room temperature (˜25° C.) for five days. After incubation, the spores were collected with 0.1% Tween 80 buffer and filtered through four layers of Miracloth into a 50-mL Falcon tube. The concentration of spores was first determined by counting the spores in a hemacytometer under the microscope and then diluted to 10⁵ conidia/mL with PBS. In a 24-well plate (4 rows×6 columns), each column was loaded with the same PDB media that had been reconstituted with a corresponding fraction of NPc. For each row, the same strain of fungus was inoculated. For each well, 1.5 mL of the corresponding media was loaded, and 15 μL of the adjusted spore suspension was delivered to make the concentrate of fungal spore approximately 10³ conidia/mL. The 24-well plate inoculated with A. fumigatus strains was incubated at 37° C. for three days, and the plate inoculated with Penicillium spp. was incubated at room temperature (˜25° C.) for five days. Photos were taken after the incubation period.

TABLE 6
Compositions of the broth media used to
test the antifungal activity of NPc

| Treatment | Composition of the broth medium |
|---|---|
| 0% NPc | 100 mL RO water + 2.4 g potato dextrose broth powder |
| 20% NPc | 80 mL RO water + 20 mL NPc + 2.4 g potato dextrose |
| | broth powder |
| 40% NPc | 60 mL RO water + 40 mL NPc + 2.4 g potato dextrose |
| | broth powder |
| 60% NPc | 40 mL RO water + 60 mL NPc + 2.4 g potato dextrose |
| | broth powder |
| 80% NPc | 20 mL RO water + 80 mL NPc + 2.4 g potato dextrose |
| | broth powder |
| 100% NPc | 100 mL NPc + 2.4 g potato dextrose broth powder |

TABLE 7
Fungal strains tested in the NPc-reconstituted broth media

| Aspergillus fumigatus strains | Penicillium species/strains |
|---|---|
| Af293 | P. commune 162_3FA |
| CEA-10 | P. chrysogenum Y1 |
| CEA-17 | P. roqueforti |
| F16216 | P. expansum PE-21 |

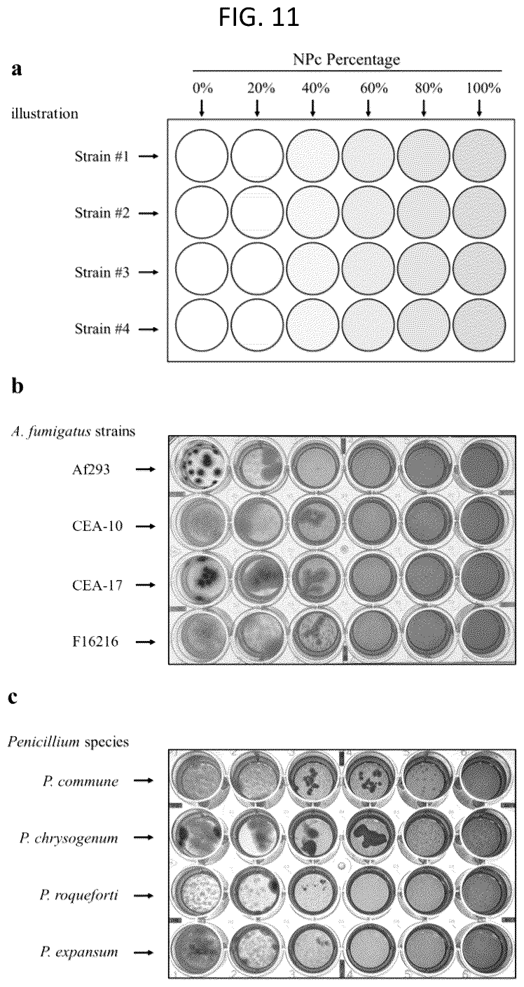

Results: No germination of spores can be visually observed for A. fumigatus strains CEA-10, CEA-17, F16216, and Af293, at a concentration of NPc as low as 60% v/v in the solvent for PDB nutrient powder (FIG. 11b). Growths of Penicillium species including P. roqueforti, P. expansum, P. commune, and P. chrysogenum were more or less inhibited by the presence of NPc (FIG. 11c).

### Antibacterial/Antifungal Activities of Ethyl Acetate Extracts From NPc and NPy

NPc and NPy can be extracted with organic solvents, including but not limited to chloroform, dichloromethane, and ethyl acetate. Given that ethyl acetate has less toxicity, it was chosen as the organic phase for NPc and NPy extractions. Two liquid-liquid extraction methods were used. In the first method, NPc or NPy was mixed with equal volumes of ethyl acetate on a shaker overnight (>8 hours). Later, we found that the acidification of NPc or NPy allowed faster and more efficient extraction. Thus, the second method involved acidifying NPc or NPy to a pH of 3-5 and then extracting with equal volumes of ethyl acetate by mixing on a vortex shaker for 30 seconds. After extraction, the organic phase was separated from the aqueous phase by centrifugation at 5,000 rpm and then evaporated under an airflow until dry. All the disk diffusion assays shown in this section were done by using the second extraction method.

To evaluate the antibacterial and antifungal activities of the extracts, we used a disk diffusion assay. First, we dissolved the extracts in methanol to a concentration reflecting 20× of the antimicrobial strength as compared to the original NPc or NPy (e.g., extracts made from 20 mL of NPc or NPy were dissolved in 1 mL of methanol). Then, we pipetted 50 μL of the re-dissolved extract solution onto a 6-mm paper disk. In this experiment, we expanded the list of microbial species and strains to test the antimicrobial spectrum of NPc and NPy, including two ESKAPE bacterial pathogens (Acinetobacter baumannii and Enterococcus faecium), an acne-causing bacterium (Cultibacterium acnes), six Listeria monocytogenes strains (10403S, LM108, LM301, LM310, R2-500, R2-501), five food spoilage yeasts (Wickerhamomyces anomalus, Pichia kudriavzevii, Clavispora lusitaniae, Debaryomyces hansenii, and Kluyveromyces marxianus), eight strains of Aspergillus fumigatus including five standard strains (Af293, CEA-10, CEA-17, F16216, and F11628), and three triazole resistant strains (1077391, 1077392, 1077395).

Results: The results of the aforementioned disk diffusion assays are shown in Table 8, except for A. fumigatus strains for which the data are shown in FIG. 12. NPc and NPy showed variable inhibitory effects on different bacterial and fungal strains. However, not a single strain showed resistance to extract made from NPc or NPy, including the ESKAPE pathogens and azole-resistant fungi. Thus, the extracts have broad-spectrum antimicrobial activities and offer great potential for use as preservatives, therapeutics, and cosmetics.

While these results were achieved with extracts of NPc and NPy, we believe that non-extracted NPc and NPy would also have antimicrobial activity against these pathogens. Extraction increases the concentration of fermentate (in this case by 20×). Thus, if necessary, it would likely be possible to increase the activity of non-extracted fermentates by simply concentrating them (e.g., by evaporating off water or drying them into powders).

TABLE 8
Microbe strains assessed by disk diffusion assays

| | | NPc zone of | NPy zone of |
|---|---|---|---|
| | | inhibition | inhibition |
| Species | Strain | diameter (mm) | diameter (mm) |
| A. baumannii | NRRL B-65371 | 17 | 18 |
| C. acnes | NRRL B-4224 | 21 | 26 |
| E. faecium | NRRL B-41204 | 14 | 17 |
| W. anomalus | H. a. 7-10-7 | 22 | 28 |
| K. marxianus | NRRL Y-2415 | 28 | 25 |
| P. kudriavzevii | NRRL Y-10940 | 14 | 15 |
| C. lusitaniae | C. l. 7-10-8 | 19 | 20 |
| D. hansenii | D. h. 7-10-9 | 33 | 31 |
| L. monocytogenes | 10403S | 15 | 15 |
| L. monocytogenes | LM108 | 14 | 16 |
| L. monocytogenes | LM301 | 15 | 15 |
| L. monocytogenes | LM310 | 15 | 15 |
| L. monocytogenes | R2-500 | 15 | 15 |
| L. monocytogenes | R2-501 | 14 | 15 |

### Conclusion

NPc and NPy fermentates derived from A. oryzae NRRL 3483 possessed potent antimicrobial activities. Hitherto, 16 different batches of both NPc and NPy have been produced and tested for their antimicrobial activity using the quality check method with 100 μL of the overnight extract. NPc generated zones of clearance with an average diameter of 18.75 mm with a standard deviation of 1.69 mm. NPy generated zones of clearance with an average diameter of 20.47 mm with a standard deviation of 1.92 mm. All the quality check data are shown in FIG. 13.

### Example 3: Various Strains of Aspergillus oryzae can be Used to Produce NPy

Different strains of Aspergillus oryzae were used to produce NPy fermentate to determine which strains produce antimicrobial effects. The A. oryzae strains used to produce NPy are listed in Table 9. The optimized fermentation parameters described in Example 1 were used. Briefly, the fermentation substrate was 10 g/L yeast extract in RO water, with each 250-mL containing 150 mL of the substrate. For each strain, one flask of content was inoculated to a level of 10⁴ conidia of the corresponding A. oryzae strain per mL of the substrate. The shaking incubator was set at 30° C. and 220 rpm agitation. All the flasks were harvested after 6 days of incubation, and the culture was filtered through glass fiber filter and collected into falcon tubes. Three biological replicates of the fermentates were collected.

TABLE 9
Aspergillus oryzae strains used to produce NPy fermentates
Aspergillus oryzae strains used in the first screen

| | | |
|---|---|---|
| NRRL 451 | NRRL 456 | NRRL 552 |
| NRRL 1808 | NRRL 2220 | NRRL 3483 |
| NRRL 3484 | NRRL 3486 | NRRL 3487 |
| NRRL 3488 | NRRL 5030 | NRRL 5938 |
| NRRL 6574 | NRRL 13765 | NRRL 32657 |

The antimicrobial activities of NPy produced using the different A. oryzae strains were assessed via a disk diffusion assay against S. aureus S-6 using 100 μL of 20× extract of NPy fermentates on 6-mm antimicrobial susceptibility disks (n=3). The experiment was done in biological triplicate. Only three out of 15 A. oryzae strains produced an antimicrobial fermentate; the rest of the strains did not generate a zone of clearance in disk diffusion assays. A. oryzae NRRL 3483 and A. oryzae NRRL 3484 generated similar sizes of zones of clearance (Table 10). A. oryzae NRRL 32657 was also found to generate a zone of inhibition, but the size was much smaller compared to those of strains NRRL 3483 and NRRL 3484 (Table 10).

TABLE 10
Results of a disk diffusion assay of overnight extracts against
S. aureus S-6 testing the antimicrobial activity of NPy fermentates
generated with 15 different A. oryzae strains

| Aspergillus oryzae NRRL | Zone of inhibition |
|---|---|
| strain number | diameter (mm) |
| 3483 | 21.3 |
| 3484 | 21.3 |
| 32657 | 14.3 |
| 451 | No Zone of Inhibition |
| 456 | No Zone of Inhibition |
| 552 | No Zone of Inhibition |
| 1808 | No Zone of Inhibition |
| 2220 | No Zone of Inhibition |
| 3486 | No Zone of Inhibition |
| 3487 | No Zone of Inhibition |
| 3488 | No Zone of Inhibition |
| 5030 | No Zone of Inhibition |
| 5938 | No Zone of Inhibition |
| 6574 | No Zone of Inhibition |
| 13765 | No Zone of Inhibition |

Given the observed variability in antimicrobial production among different A. oryzae strains, we expanded our strain panel and employed an acidified extraction method to prepare ethyl acetate extracts from their respective NPy fermentates. These extracts were then tested using 50-μL disk diffusion assays against Staphylococcus aureus S-6. Table 11 summarizes the results for the additional A. oryzae strains that exhibited antimicrobial activity. The observed differences in inhibition zones among strains indicate that antimicrobial production is strain-dependent.

While these results were achieved with extracts of NPy, we believe that non-extracted NPy produced using these additional A. oryzae strains would also have antimicrobial activity. Extraction increases the concentration of fermentate (in this case by 20×). Thus, if necessary, it would likely be possible to increase the activity of non-extracted fermentates by simply concentrating them (e.g., by evaporating off water or drying them into powders).

TABLE 11
Results of disk diffusion assay evaluating the antimicrobial
activity of ethyl acetate extracts derived from acidified NPy fermentates
produced by different Aspergillus oryzae strains against
Staphylococcus aureus S-6. Inhibition zones were measured
to assess the relative efficacy of each extract.

| Aspergillus oryzae strain number | Zone of inhibition diameter (mm) |
|---|---|
| NRRL 506 (same as NRRL 1653) | 22 |
| NRRL 6270 | 23 |
| KACC SD20 | 18 |
| KACC 46922 | 27 |
| KACC Aor44 | 17 |
| KACC SD45 | 16 |
| Han AO097 | 25 |
| KACC 46923 | 18 |
| KACC Meju 529 | 20 |
| Han #6 | 19 |
| KACC 46465 | 25 |
| KACC 46456 | 26 |
| KACC 46457 | 23 |
| KACC 93120 (M385) | 11 |
| KACC 46466 | 24 |
| KACC 46920 | 28 |
| KACC 46891 | 29 |
| Han AO2014 | 20 |
| Han #334 | 20 |
| KACC 46810 | 25 |
| KACC 46468 | 21 |
| China 3.267 | 17 |
| China 3.47 | 11 |
| KACC M1017 | 17 |
| Han #266 | 14 |
| Han #17 | 13 |
| Han #13 | 21 |

### Example 4: Various Substrates can be Used to Produce Antimicrobial NP Fermentates

### Production of Antimicrobial Fermentate Using Egg White Protein Solution as the Substrate

Given that NPc and NPy are both derived from substrates containing amino acids as the major nutrient, we attempted to use dried egg white powder (i.e., a food-grade protein substrate) to produce an antimicrobial NP fermentate, which is hereafter referred to as “NPew.” The fermentation parameters and process for producing NPew were all the same as those used to produce NPy (see Example 1), except that the fermentation substrate was 10 g/L egg white protein solution, which was made by adding 10 grams of egg white protein powder (XPRS Nutra) into one liter of RO water. The antimicrobial activity was checked by disk diffusion assay against S. aureus S-6.

Results: The antimicrobial strength of the NPew fermentate was comparable to those of NPc and NPy as assessed by disk diffusion assay against S. aureus S-6 (FIG. 14). This finding suggests that egg white protein can also be a substrate for the production of an antimicrobial fermentate, which suggests that there is potential for using other food products as the substrate for generating antimicrobial NP fermentate.

### Production of Antimicrobial Fermentates Using Other Food-Grade Substrates

Different food-grade substrates were screened as fermentation substrates to produce antimicrobial NP fermentates. The tested substrates are listed in Table 12. The fermentation parameters and process for producing these NP fermentates were all the same as those used to produce NPy (see Example 1), except that the fermentation substrate was a 20 g/L solution of the corresponding food-grade powdered ingredients. The antimicrobial activity of the fermentates was checked by disk diffusion assay against S. aureus S-6.

TABLE 12
Food-grade substrates tested for their
ability to produce NP fermentates

| Product Name | Brand |
|---|---|
| Raw Organic Protein, Plant-based | Garden of Life LLC |
| Protein Warrior Blend, Plant-based | Sunwarrior |
| Whey Protein Concentrate | nutricost |
| Soy Protein Isolate (Refined & Soluble) | Pure Original Ingredients |
| Micronized Creatine Monohydrate | nutricost |
| Branched Chain Amino Acids | nutricost |
| Essential Amino Acids | nutricost |
| Hydrolyzed Bone Broth Protein | Zammex Nutrition LLC |
| Infant Formula Gerber Extensive HA | Nestle Infant Nutrition |
| Naked Micellar Casein | Naked Nutrition |

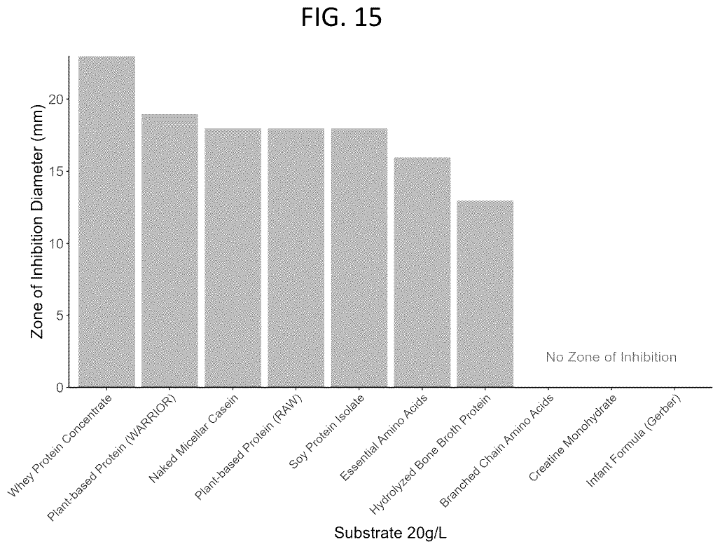

Results: All the tested substrates except branched chain amino acids, creatine monohydrate, and infant formula, generated antimicrobial activity (FIG. 15). Based on this screening, the fermentate derived from whey protein concentrate possessed the highest level of antimicrobial activity, followed by Warrior brand plant-based protein mix, micellar casein, Raw brand plant-based protein mix, soy protein isolate, essential amino acids, and hydrolyzed bone broth. Of all these antimicrobial fermentates, only hydrolyzed bone broth yielded a relatively small zone of clearance (13 mm), whereas the fermentates from other products generated zones of clearance comparable in size to those from NPc and NPy. This suggests that a wide range of substrates containing proteins or amino acids can be used with A. oryzae strains to generate antimicrobial NP fermentates.

### Example 5: Evaluation of the Abilities of Three Aspergillus oryzae Strains to Produce Antimicrobial Activities in NPc, NPy, and NPew

In view of the finding that, in addition to A. oryzae NRRL 3483, A. oryzae NRRL 32657 can also be used to produce an antimicrobial NPy fermentate (Example 3), we were interested to know whether A. oryzae strain NRRL 32657 can be used to produce antimicrobial activity also in Npew. Thus, we evaluated the ability of this strain, along with A. oryzae NRRL 3483 and A. oryzae NRRL 3484 for comparison, to produce NPc, NPy, and NPew. The fermentates were produced as described in Example 1 and Example 4, except that the A. oryzae strain that was inoculated was varied. NPc, NPy, and NPew were produced for each of the three strains. Upon harvesting the NP fermentates, the antimicrobial activities were checked via disk diffusion assay against S. aureus S-6.

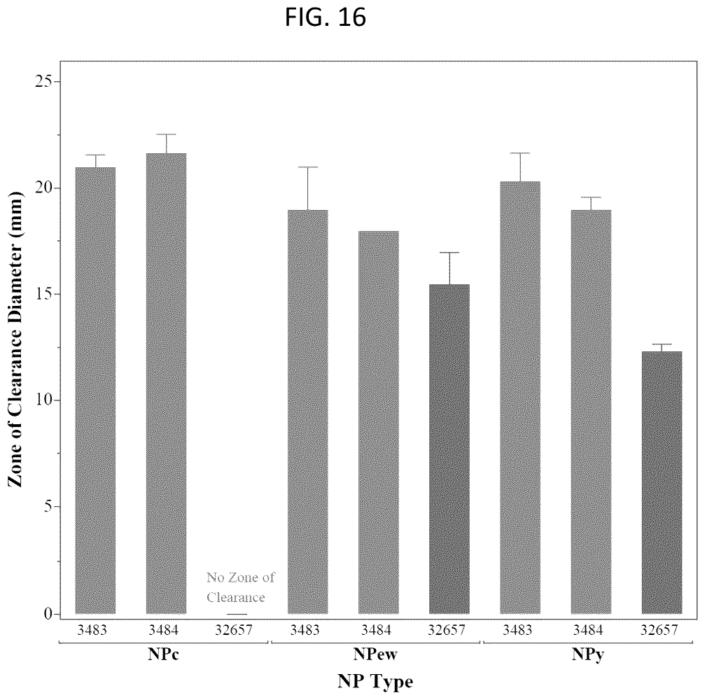

The A. oryzae strains NRRL 3483 and NRRL 3484 consistently produced strong antimicrobial activities in NPc, NPy, and NPew (FIG. 16). However, A. oryzae strain NRRL 32657 produced little or no antimicrobial activity in NPc as there was no zone of clearance detected for its extract (FIG. 16). The NPy produced by A. oryzae NRRL 32657 showed lower levels of antimicrobial activity compared to the other two strains, which is consistent with the results discussed in Example 3. Similarly, A. oryzae NRRL 32657 also produced slightly lower levels of antimicrobial activity compared to the other two strains in NPew, although the differences of the zone of clearance diameters among the three strains were less compared to NPc and NPy.

### Example 6: Heat Stability of the Antimicrobial Activity of NPc, NPy, and NPew

The heat stabilities of the antimicrobial activities in NPc, NPy, and NPew were validated by comparing disk diffusion assay results between filter-sterilized samples (non-heat-treated) and autoclaved (15 psig for 20 minutes at 121° C.) samples (heat-treated). NPc, NPy, and NPew were produced as described in Example 1 and Example 4, using the same fermentation parameters and production steps. Antimicrobial activity was assessed using a disk diffusion assay with 100 μL of 20× extract against S. aureus S-6. The experiment was done in biological triplicate.

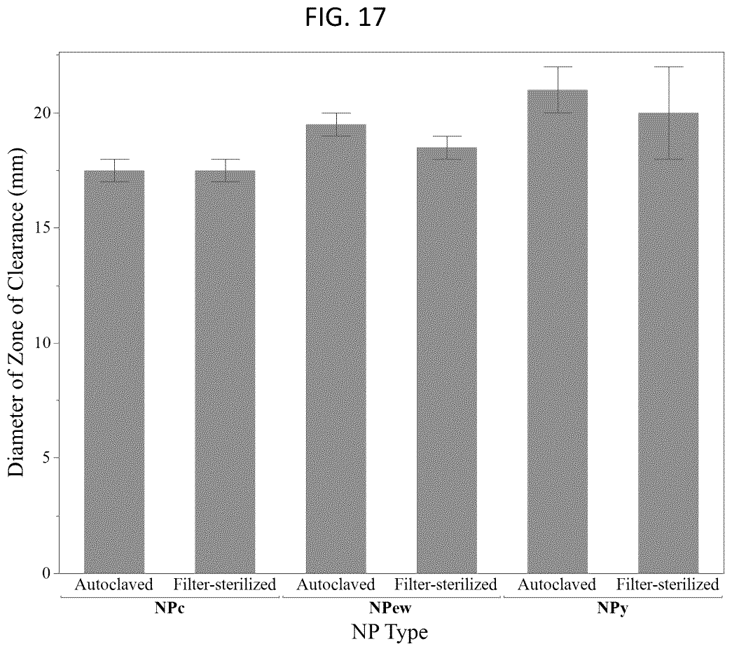

Results: There were no significant differences found in the sizes of the zones of clearance between the pair of filter-sterilized and autoclaved samples for NPc, NPy, and NPew (FIG. 17), suggesting that the antimicrobial component(s) in NP fermentates are stable up to at least 121° C. The thermal stabilities of the NP fermentates enable their applications in various food manufacturing processes.

### Example 7: Characterization of the Chemical Composition of NPc and NPy

As we sought to develop NPs as natural, clean-label food additives, it is critical to understand the chemical composition of NPs. This analysis was done prior to the RSM optimization of NPy fermentation parameters, so NPy was fermented with 20 g/L yeast extract as the substrate and 10⁵ conidia/mL as the inoculation level. The fermentates were sent to Eurofins (USA) for the analyses.

Results: All screened compounds were below the detection thresholds except for some amino acids and zinc (Table 13). As the fermentation substrates (i.e., pancreatic digest of casein and yeast extract) primarily contain amino acids and peptides, it was expected that amino acids would be left in the NP fermentates. Given that NPc and NPy have a clean chemical profile, we expect that the addition of NPc and NPy will not significantly impact the nutritional value and the texture of foods, allowing these fermentates to be used in a variety of different food types.

TABLE 13
The chemical composition of NPc and NPy fermentates

| | | |
|---|---|---|
| Analyte | NPc | NPy^{†} |

Fatty Acids Calculated as
Triglycerides (Method: FALT_S)

| | | |
|---|---|---|
| Saturated Fatty Acids (Acid Form) | <0.002% | <0.002% |
| Total Cis Unsaturated Fatty Acids | <0.002% | <0.002% |
| (Acid Form) | | |
| Monounsaturated Fatty Acids | <0.002% | <0.002% |
| (Acid Form) | | |
| Polyunsaturated Fatty Acids | <0.002% | <0.002% |
| (Acid Form) | | |
| Trans Fatty Acids (Acid Form) | <0.002% | <0.002% |
| Omega 3 Fatty Acids | <0.002% | <0.002% |
| Omega 6 Fatty Acids | <0.002% | <0.002% |
| Omega 9 Fatty Acids | <0.002% | <0.002% |
| Total Fatty Acids | <0.002% | <0.002% |
| 4:0 Butyric | <0.002% | <0.002% |
| 6:0 Caproic | <0.002% | <0.002% |
| 8:0 Caprylic | <0.002% | <0.002% |
| 10:0 Capric | <0.002% | <0.002% |
| 12:0 Lauric | <0.002% | <0.002% |
| 14:0 Myristic | <0.002% | <0.002% |
| 14:1 Myristoleic | <0.002% | <0.002% |
| 15:0 Pentadecanoic | <0.002% | <0.002% |
| 15:1 Pentadecenoic | <0.002% | <0.002% |
| 16:0 Palmitic | <0.002% | <0.002% |
| 16:1 Palmitoleic | <0.002% | <0.002% |
| 17:0 Heptadecanoic | <0.002% | <0.002% |
| 17:1 Heptadecenoic | <0.002% | <0.002% |
| 18:0 Stearic | <0.002% | <0.002% |
| 9c 18:1 Oleic | <0.002% | <0.002% |
| 18:2 Linoleic | <0.002% | <0.002% |
| 18:3 Gamma Linolenic | <0.002% | <0.002% |
| 18:3 Alpha Linolenic | <0.002% | <0.002% |
| 18:4 Octadecatetraenoic | <0.002% | <0.002% |
| 20:0 Arachidic | <0.002% | <0.002% |
| 20:1 Eicosenoic | <0.002% | <0.002% |
| 20:2 Eicosadienoic | <0.002% | <0.002% |
| 20:3 Eicosatrienoic (n3) | <0.002% | <0.002% |
| 20:3 Homogamma Linolenic (n6) | <0.002% | <0.002% |
| 20:4 Arachidonic (n3) | <0.002% | <0.002% |
| 20:4 Arachidonic (n6) | <0.002% | <0.002% |
| 20:5 Eicosapentaenoic | <0.002% | <0.002% |
| 21:5 Heneicosapentaenoic | <0.002% | <0.002% |
| 22:0 Behenic | <0.002% | <0.002% |
| 22:1 Erucic | <0.002% | <0.002% |
| 22:2 Docosadienoic | <0.002% | <0.002% |
| 22:3 Docosatrienoic | <0.002% | <0.002% |
| 22:4 Docosatetraenoic | <0.002% | <0.002% |
| 22:5 Docosapentaenoic (n3) | <0.002% | <0.002% |
| 22:5 Docosapentaenoic (n6) | <0.002% | <0.002% |
| 22:6 Docosahexaenoic | <0.002% | <0.002% |
| 24:0 Lignoceric | <0.002% | <0.002% |
| 24:1 Nervonic | <0.002% | <0.002% |
| Total 18:1 trans | <0.002% | <0.002% |
| Total 18:1 cis | <0.002% | <0.002% |
| Total 18:2 trans | <0.002% | <0.002% |
| Total 18:3 trans | <0.002% | <0.002% |

Sugar Profile by Ion Chromatography
(Method: SGIC_2_S)

| | | |
|---|---|---|
| Fructose | <0.1% | <0.1% |
| Galactose | <0.1% | <0.1% |
| Glucose | <0.1% | <0.1% |
| Sucrose | <0.1% | <0.1% |
| Lactose | <0.1% | <0.1% |
| Isomaltulose | <0.1% | <0.1% |
| Maltose | <0.1% | <0.1% |
| Total Sugar | <0.1% | <0.1% |

Vitamin C (Method:
AOACVITC_S)

| | | |
|---|---|---|
| Vitamin C | <0.00248% | <0.00248% |

Thiamin by Fluorometric Method
(Method: BIDE_S)

| | | |
|---|---|---|
| Thiamin | 0% | 0% |

Biotin by Microbiological Method
(Method: BIOM_S)

| | | |
|---|---|---|
| Biotin | 0% | 0% |

Amino Acids (Method:
TAALC_S)

| | | |
|---|---|---|
| Aspartic Acid | 0.105% | 0.0490% |
| Threonine | 0.0449% | 0.0108% |
| Serine | 0.0661% | 0.0106% |
| Glutamic Acid | 0.312% | 0.0871% |
| Proline | 0.112% | 0.0125% |
| Glycine | 0.0234% | 0.0172% |
| Alanine | 0.0247% | <0.0100% |
| Valine | 0.0456% | <0.0100% |
| Isoleucine | 0.0415% | <0.0100% |
| Leucine | 0.0321% | <0.0100% |
| Tyrosine | 0.0118% | <0.0100% |
| Phenylalanine | 0.0170% | <0.0100% |
| Lysine | 0.0487% | 0.0195% |
| Histidine | 0.0279% | <0.0100% |
| Arginine | <0.0100% | 0.0102% |
| Cystine | <0.0100% | <0.0100% |
| Methionine | 0.0190% | <0.0100% |
| Collagen (Calculated from | <0.0800% | <0.0800% |
| hydroxyproline using a conversion | | |
| factor of 8) | | |

Organic Acids (Method: ORG4_S)

| | | |
|---|---|---|
| Propionic Acid | <0.0400% | <0.0400% |

Organic Acids: Benzoic and Sorbic Acids
(Method: ORG1_S)

| | | |
|---|---|---|
| Benzoic Acid | <0.000400% | <0.000400% |
| Sorbic Acid | <0.000400% | <0.000400% |

Total Polyphenols (Method: TOTP_S)

| | | |
|---|---|---|
| Total Polyphenols (Gallic | <0.0150% | <0.0150% |
| Acid Equivalents) | | |

Elements by ICP Mass Spectrometry
(Method: ICP_MS_S)

| | | |
|---|---|---|
| Zinc | 0.000104% | 0.000214% |

^{†}Both NPc and NPy were produced with 20 g/L of substrate, 10⁵ conidia/mL of A. oryzae NRRL 3483 spores as the inoculation level, and 6 days of incubation at 30° C. and 220 rpm agitation.

### Example 8: Food Applications of NPs

### Cheese Coating Solutions Made From NPs

Given the broad-spectrum antimicrobial activities of NPs as well as their food-grade identities, we aimed to transform these antimicrobial compositions into food preservatives. The first product we designed was a cheese coating solution. It was made by adding 0.5% w/v xanthan gum into filtered (but not extracted) NPc or NPy and mixing with mild heating to dissolve the gum fully. The thickened NPc or NPy can then be applied onto cheese surfaces by dipping the cheese slice into the solution. After air-drying the surface, we examined the antimicrobial activity of the coating by spreading L. monocytogenes (5.5 log CFU/g cheese) or spotting 100 Penicillium spores at the center of the cheese.

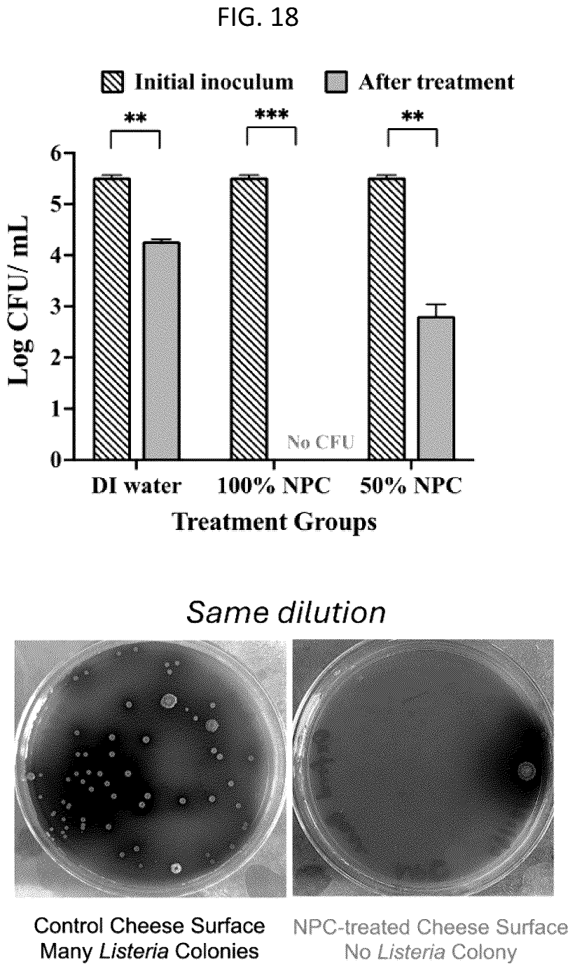

For cheese inoculated with L. monocytogenes, the cheese was stored at 4° C. for 24 hours, and the number of L. monocytogenes was assessed by the plate-count method. With 100% NPc cheese coating, we observed no colonies of L. monocytogenes, suggesting that the cheese coating can kill L. monocytogenes (FIG. 18). For cheese spotted with Penicillium spores, we observed that both NPc and NPy cheese coatings can effectively inhibit the growth of Penicillium spp. at both 4° C. (refrigeration storage temperature) and 12° C. (cheese-aging temperature) (FIG. 19). Therefore, NP cheese coating has the dual effect of antibacterial and antifungal properties, making it a promising clean-label solution to prevent microbial contamination in cheese products.

### Freeze-Dried NPc as a Yogurt Preservative

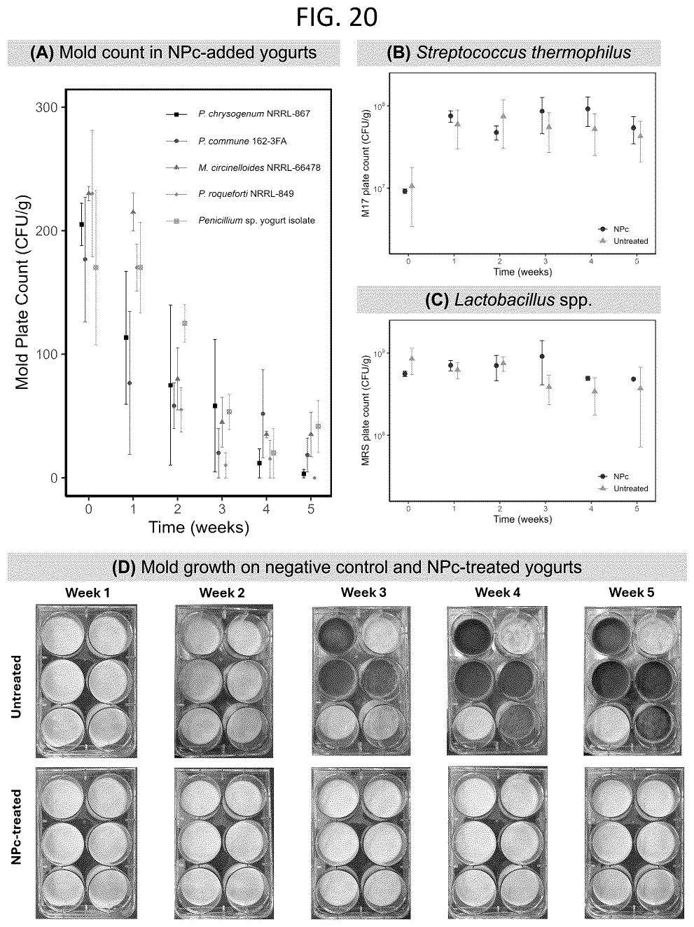

Yogurt is another food that can be easily contaminated with molds and/or yeast. To address this problem, we freeze-dried NPc into powders and added the powder to yogurt at a concentration of ˜1.1% w/w. To assess its efficacy, we inoculated 1,000 mold spores per gram of yogurt, stored at 4° C., and tracked the number of mold colonies every week by plate count. Served for a visual comparison, yogurt without NPc addition was also inoculated with mold. In addition, to evaluate whether NP impacts the lactic acid bacteria in yogurt, we also tracked on the number of Streptococcus thermophilus and Lactobacilli, on M17 and MRS agars, respectively. In NPc-added yogurt, the plate counts for five different molds all decreased (FIG. 20A). No visual appearance changes were observed for NPc-added yogurt, while the negative control yogurt had its surface fully occupied by inoculated molds (FIG. 20D). Surprisingly, the counts of S. thermophilus and Lactobacilli were not significantly different between negative control and NPc-treated yogurt, suggesting that NPc may not interfere with the lactic acid bacteria and probiotics in yogurt drink (FIG. 20 B & C). We also did a preliminary antimicrobial test against yeasts (Wickerhamomyces anomalus, Pichia kudriavzevii (also known as Candida krusei), Candida lusitaniae, Debaryomyces hanseni, and Kluyveromyces marxianus) in negative control yogurt and NPc-yogurt, and we observed that, at 10° C., the negative control yogurt became coagulated with texture changes, while NPc-yogurt remained smooth in texture (intact).

What is claimed:

1. A method of producing an antimicrobial composition, the method comprising:
a) inoculating a medium with an Aspergillus oryzae strain to produce an inoculated medium; and
b) culturing the inoculated medium to produce a fermentate;
wherein the medium comprises a source of amino acids at a concentration of 3-40 g/L, and
wherein the medium does not comprise casein.

2. The method of claim 1, further comprising:
c) filtering the fermentate to obtain a cell-free fermentate filtrate.

3. The method of claim 2, further comprising:
d) extracting the cell-free fermentate filtrate to obtain an extract of the cell-free fermentate filtrate.

4. The method of claim 1, wherein the source of amino acids is yeast extract, egg white, whey protein, a plant-based protein mixture, soy protein isolate, essential amino acids, or hydrolyzed bone broth protein.

5. The method of claim 1, wherein the Aspergillus oryzae strain is NRRL 3483, NRRL 3484, NRRL 32657, NRRL 506, NRRL 6270, KACC SD20, KACC 46922, KACC Aor44, KACC SD45, Han AO097, KACC 46923, KACC Meju 529, Han #6, KACC 46465, KACC 46456, KACC 46457, KACC 93120 (M385), KACC 46466, KACC 46920, KACC 46891, Han AO2014, Han #334, KACC 46810, KACC 46468, China 3.267, China 3.47, KACC M1017, Han #266, Han #17, or Han #13.

6. The method of claim 1, wherein:
i) the medium is inoculated with 10³ to 10⁷ conidia/mL of the Aspergillus oryzae strain,
ii) the inoculated medium is cultured for 2 to 10 days,
iii) the inoculated medium is cultured at 22 to 30° C.,
iv) the inoculated medium is cultured with shaking at 150 to 220 rpm, or
v) any combination thereof.

7. The method of claim 6, wherein the inoculated medium is cultured for 6-7 days.

8. An antimicrobial composition produced by the method of claim 1.

9. The antimicrobial composition of claim 8, wherein the composition has antibacterial activity against one or more bacteria selected from Staphylococcus aureus, Escherichia coli, Klebsiella pneumoniae, Pseudomonas aeruginosa, Salmonella enterica, Listeria monocytogenes, Acinetobacter baumannii, Cutibacterium acnes, and Enterococcus faecium.

10. The antimicrobial composition of claim 8, wherein the composition has antifungal activity against one or more fungi selected from Candida albicans, Penicillium roqueforti, Penicillium expansum, Penicillium camemberti, Penicillium chrysogenum, Penicillium commune, Aspergillus fumigatus, Wickerhamomyces anomalus, Kluyveromyces marxianus, Pichia kudriavzevii, Candida lusitaniae, Clavispora lusitaniae, and Debaryomyces hansenii.

11. The antimicrobial composition of claim 8, wherein the composition is formulated for oral or topical administration.

12. The antimicrobial composition of claim 11, wherein the composition is formulated as a food additive.

13. A product comprising the antimicrobial composition of claim 8.

14. The product of claim 13, wherein the product is a food product.

15. The product of claim 14, wherein the food product is cheese or yogurt.

16. A method of inhibiting the growth of a microbe in or on a food product, the method comprising: applying the antimicrobial composition of claim 8 to the food product.

17. A method of inhibiting the growth of a microbe on a surface, the method comprising: applying the antimicrobial composition of claim 8 to the surface.

18. A method of inhibiting the growth of a microbe in or on a subject, the method comprising: administering the antimicrobial composition of claim 8 to the subject.

19. A method of using an Aspergillus oryzae fermentate composition, the method comprising: applying or administering the composition to a subject in need thereof.

20. The method of claim 19, wherein the subject is in need of hair growth, prevention of hair loss, amelioration of dandruff, treatment for acne, skin tone or color maintenance.
